SEQUENCE LISTING (24 sequences)

SEQ ID NO: 1 - protein, 324 aa, Homo sapiens
MSDLIVKDNALMNASYNLALVEQRLILLAIIEARETGKGINANDPLTVHASSYINQFNVERHTAYQALKDACKDLFARQFSYQEKRERGRINITSRWVSQIGYMDDTATVEIIFAPAVVPLITRLEEQFTQYDIEQISGLSSAYAVRMYELLICWRSTGKTPIIELDEFRKRIGVLDTEYTRTDNLKMRVIELALKQINEHTDITASYEQHKKGRVITGFSFKFKHKKQNSDKTPKNSDSSPRIVKHSQIPTNIVKQPENAKMSDLEHRASRVTGEIMRNRLSDRFKQGDESAIDMMKRIQSEIITDAIADQWESKLEEFGVVF

SEQ ID NO: 2 - protein, 18 aa, Homo sapiens
EARETGKGINANDPLTVH

SEQ ID NO: 3 - protein, 21 aa, Homo sapiens
KQINEHTDITASYEQHKKGRT

SEQ ID NO: 4 - protein, 8 aa, synthetic construct
GAHHHHHH

SEQ ID NO: 5 - protein, 11 aa, synthetic construct
MASMTGGQQMG

SEQ ID NO: 6 - protein, 8 aa, synthetic construct
DYKDDDDK

SEQ ID NO: 7 - protein, 8 aa, synthetic construct
WSHPQFEK

SEQ ID NO: 8 - protein, 319 aa, Homo sapiens
MSKLVVKDNALMNASYNLDLVEQRLILLAIIEARESGKGINANDPLTVHAESYINQFGVHRVTAYQALKDACDNLFARQFSYQSKSEKGNIQNHRSRWVSEIIYIDTEATVKIIFAPAIVPLITRLEEQFTKYDIEQISDLSSAYAIRLYELLICWRSTGKTPIIGLGEFRNRVGVLDSEYHRIAHLKERVIEHSIKQINEHTDITATYEQHKKGRTITGFSFKFKQKKPKQAEIATETPKTATNDPDTTKPLTEPQIAKYSMILCKLGSISDLSNFPDYPAFANWIGNILRNPEKADEQIAKRIFTALKTETDYSKKN

SEQ ID NO: 9 - protein, 7 aa, Homo sapiens
NRLSDRF

SEQ ID NO: 10 - protein, 326 aa, unidentified
MSDLIVKDNALMNASYNLALVEQRLILLAILEARETGKGINANDPLTVHASSYINQFNVERHTAYQALKDACKDLFARQFSYQEKRERGRINITSRWVSQIGYMDDTATVEIIFAPAVVPLITRLEEQFTQYDIEQISELSSAYAVRLYELLICWRSTGKTPIIDLTEFRKRLGVLDTEYTRTDNLKMRVIELGLKQINEHTDITASYEQHKKGRTITGFSFKFKQKKKTGAEMPKNSDSSPHIEKPSQIPANIAKQPENAKKDDLGHRASKITGLIMSNGLADRFKRGDESVIDMMKRIKEEITTDTTADQWENKLEEFGVIFQS

SEQ ID NO: 11 - protein, 324 aa, unidentified
MSDLIVKDNALMNASYNLDLVEQRLILLAILEARETGKGINANDPLTVHAESYINQFGVARQTAYQALKDACKDLFARQFSYQEKRERGRANITSRWVSQIAYIDETATVEVIFAPAVVPLITRLEEQFTQYDIEQISGLSSAYAVRLYELLICWRSTGKTPVIELAEFRKRLGVLNDEYTRSDNFKKWIIENPIKQINEHTDITASYEQHKKGRTITGFSFKFKQKKKTEPETPKNSDSSQRIEKPSQIPANIVKQPENANLSDLQHRASKITGLIMSNRLSDRFKQGDESIMQMMARIQSEITTDSIADQWQSKLEEFGVVF

SEQ ID NO: 12 - protein, 324 aa, unidentified
MSDLIVKDNALMNASYNLALVEQRLILLAILEARETGKGINANDPLTVHASSYINQFNVERHTAYQALKDACKDLFARQFSYQEKRERGRANITSRWVSQIAYIDETATVEVIFAPAVVPLITRLEEQFTQYDIEQISGLSSAYAVRLYELLICWRTTGKTPVLDLTEFRKRLGVLDTEYTRTDNLKMRVIEQSLKQINKHTDITASYEQHKKGRTITGFSFKFKQKKKTEPETPKNNDSGVSKPKTVEIPAEVVKQPKNTNLSDLEKRVRMITGAIAKNNLASRFQHGNESPLDMMKRIQSEITSDETADLWQNKLESMGVVF

SEQ ID NO: 13 - DNA, 999 bp, unidentified
  source 1..999: mol_type = other DNA
atgagcgacctgatcgtgaaagacaatgccctgatgaacgcctcctacaacctggcactggtcgaacagagactgattctgctggctatcatcgaggcaagggagaccggcaagggcatcaacgccaatgaccccctgacagtgcacgccagctcctacatcaaccagtttaatgtggagcgccacaccgcctatcaggccctgaaggacgcctgcaaggatctgtttgcccggcagttcagctaccaggagaagcgggagagaggcaggatcaacatcacaagcagatgggtgtcccagatcggctatatggacgataccgccacagtggagatcatctttgcaccagcagtggtgcctctgatcaccaggctggaggagcagttcacacagtacgacatcgagcagatctccggactgtctagcgcctacgccgtgcgcatgtatgagctgctgatctgttggcggtctaccggcaagacacctatcatcgagctggatgagttccgcaagcggatcggcgtgctggacaccgagtacaccagaacagataacctgaagatgagagtgatcgagctggccctgaagcagatcaatgagcacaccgatatcacagcctcttatgagcagcacaagaagggccgcgtgatcaccggcttcagctttaagttcaagcacaagaagcagaactctgacaagacaccaaagaatagcgattcctctccccggatcgtgaagcacagccagatccctaccaacatcgtgaagcagccagagaatgccaagatgtccgacctggagcacagggcatctagggtgacaggcgagatcatgagaaataggctgagcgatcggttcaagcagggcgacgagtccgccatcgatatgatgaagagaatccagtccgagatcatcaccgacgccatcgccgatcagtgggaatctaaactggaagagtttggagtcgtgtttggagcacatcaccatcatcatcactga

SEQ ID NO: 14 - protein, 326 aa, Homo sapiens
MSDLIVKDNALMNASYNLALVEQRLILLAIIEARETGKGINANDPLTVHASSYINQFNVERHTAYQALKDACKDLFARQFSYQEKRERGRINITSRWVSQIGYMDDTATVEIIFAPAVVPLITRLEEQFTQYDIEQISGLSSAYAVRMYELLICWRSTGKTPIIELDEFRKRIGVLDTEYTRTDNLKMRVIELALKQINEHTDITASYEQHKKGRVITGFSFKFKHKKQNSDKTPKNSDSSPRIVKHSQIPTNIVKQPENAKMSDLEHRASRVTGEIMRNRLSDRFKQGDESAIDMMKRIQSEIITDAIADQWESKLEEFGVVFGA

SEQ ID NO: 15 - DNA, 974 bp, Homo sapiens
  source 1..974: mol_type = genomic DNA
atgagcgatttaatagtaaaagataacgccctaatgaatgctagttataacttagctttggttgaacagaggttaattctattagcaatcatagaagcgagagaaacaggcaaagggattaatgccaatgatcctttaacagttcatgcaagtagctatatcaatcaatttaacgtagaaaggcatacggcatatcaagccctcaaagatgcttgtaaagacttgtttgcccgtcaattcagttaccaagaaaagcgagaacgaggacgaattaatattacaagtcgatgggtttcgcaaattggctatatggacgatacagcaaccgttgagattatttttgcccctgcggttgttcctctgattacacggctagaggaacagttcacccagtacgatattgagcaaattagcggtttatcgagtgcatatgctgttcgtatgtacgaactgctgatttgttggcgtagcacaggcaaaacaccaattattgagctagacgagtttagaaagcgaataggtgttttagatactgaatacactagaacagataatttaaagatgcgagttattgaattagccctaaaacaaatcaacgaacatacagacatcacagcaagctatgaacaacacaaaaaagggcgagtgattacaggattctcattcaagtttaagcacaagaaacaaaacagcgataaaacgccaaaaaatagcgattctagcccacgtatcgtaaaacatagtcaaatccctccaacattgtaaaacagcctgaaaacgccaaaatgagcgatttagaacatagagcgagccgtgttacaggggaaataatgcgaaatcgtctgtcagatcggtttaaacaaggcgatgaatcagcaatcgacatgatgaaacgtattcaaagtgaaataataaccgatgcaatagcagaccagtgggaaagcaaactggaggagtttggcgtggttttttag

SEQ ID NO: 16 - protein, 327 aa, unidentified
MSDLIVKDNALMNASYNLALVEQRLILLAILEARETGKGINANDPLTVHANSYINQFNVERHTAYQALKDACKDLFARQFSYQEKRERGRINITSRWVSQIGYMDDTATVEVIFAPAVVPLITRLEEQFTQYDIEQISGLSSAYAVRLYELLICWRSTGKTPVIELAEFRKRIGVLDTEYTRTDNLKMRVIELALKQINEHTDITASYEQHKKGRTITGFSFKFKQKKKTELETPKNSDSSPHIEKPSQIPTNIVKQPENAKKDDLGHRASKITGLIMSNGLADRFKRGDESVMNMMKRIKEEITTDATADLWQNKLESMVWFFSHD

SEQ ID NO: 17 - protein, 323 aa, unidentified
MYRWFMSDLIVKDNALMNASYNLDLVEQRLVLLAILEARESGKGINANDPLTVHAESYINQFGVARQTAYQALKGACKDLFARQFSYQEKRERGRANITSRWVSQIAYIDETATVEVIFAPAVVPLITRLEEQFSKYDIEQISSLSSAYAVRLYELLICWRTTGKTPVIDLTEFRKRIGVLDTEYQRMDVFKRGVLELALKQVNEHTDITATYEQHKKGRVITGFSFKFKQKKPKQAEIATETPKTATTDLATTKPLTEPQIAKYSMILCKLGSISDLGNFPDYPTFANWIANILRTPEKADEQIAKRIFTALKTETDYSKKN

SEQ ID NO: 18 - protein, 308 aa, unidentified
MKNGLVVKDNALMNASYNLEVTEQRLILLAIISARETGQGITSDSKLEIHASDYAIQFSVAKETAYEALKSAVNNLFERQFSFREETKKGTGIVRSRWVSRIKYIDDAAILEITFAPDVVPLITRLEEHFTSYQIKQIAQLTSKYAIRLYELLIAWRTTGKVPELELSEFRNRLGIASNEYTAMNNFKSRVLEPSIKQINEHTDITVTYEQHKKGRTITGFSFRFKQKQQAKKIETNRDPNTPDFFIKMTDAQRHLFANKMSEMPEMGKYSQGTESYQQFAIRIADMLLQPEKFRELYPILEKAGFKE

SEQ ID NO: 19 - protein, 318 aa, unidentified
MSELIVKDNALMNASYNLDLVEQRLILLAILEARESGKGINANDPLTVHAESYINQFGVHRTTAYQALKDACKDLFARQFSYQEKRERGRANITSRWVSQIAYIDETGTVEVIFAPAVVPLITRLEEQFTKYDIEQISGLSSAYAVRLYELLICWRTTGKTPIIDLTEFRKRIGVLDTEYQRMDVFKRGVLELALKQVNEHTDITASYEQHKKGRVITGFSFKFKQKKPKQAEIATETPKTATNDPDTTKPLTEPQIAKYSMILCKLGSISDLSNFPDYPAFANWIGNILRNPEKADEQIAKRIFTALKTETDYSKKN

SEQ ID NO: 20 - protein, 322 aa, unidentified
MRLMSELVVKDNALMNASYNLDLVEQRLILLAIVEARESGKGINANDPLTVHAESYINQFGVARQTAYQALKDACDDLFARQFSYQEINERGNTENVRSRWVSEVRYVDAEATVKLIFAPIVIPLITRLEERFTQYEMKQISELSTGYAIRLYELLICWRATGKTPVIELADFRQRMGVLDSEYQRMYDLKKYVLEPSLKQINEHTDITATYEQHKKGRVITGFSFKFKQKKPKQAEIATETPKTATNDQNTIKPLTEPQIAKYSMILCKLGSISDLSNFPDYPSFANWIANILRTPEKADEQAAKRIFTALKTETDYSKKN

SEQ ID NO: 21 - DNA, 33 bp, synthetic construct
  source 1..33: mol_type = other DNA
gcggatccgccatgagcgacctgatcgtgaaag

SEQ ID NO: 22 - DNA, 33 bp, synthetic construct
  source 1..33: mol_type = other DNA
gcggtacctcaaaacacgactccaaactcttcc

SEQ ID NO: 23 - DNA, 112 bp, synthetic construct
  source 1..112: mol_type = other DNA
gtggatccgccatggactacaaagaccatgacggtgattataaagatcatgacatcgattacaaggatgacgatgacaagggagcaagcgacctgatcgtgaaagacaatgc

SEQ ID NO: 24 - DNA, 39 bp, synthetic construct
  source 1..39: mol_type = other DNA
gaggtacctcaaaacacgactcaaactcttccagtttag

(19) United States
(12) Patent Application Publication
(10) Pub. No.: US 2025/0067744 A1
(43) Pub. Date: Feb. 27, 2025

(54) USE OF BMMF1 REP PROTEIN AS DIAGNOSTIC MARKER FOR PANCREATIC CANCER AND DIABETES MELLITUS TYPE 2

(71) Applicant: Deutsches Krebsforschungszentrum Stiftung des öffentlichen Rechts, Heidelberg (DE)

(72) Inventors: Timo Bund, Heidelberg (DE); Harald Zur Hausen, Heidelberg (DE); Ethel-Michele DE VILLIERS-ZUR HAUSEN, Heidelberg (DE); Kristina Alikhanyan, Heidelberg (DE); Claudia Ernst, Heidelberg (DE); Claudia Tessmer, Heidelberg (DE); Nathalia Giese, Schriesheim (DE)

(21) Appl. No.: 18/942,073

(22) Filed: Nov. 8, 2024

Related U.S. Application Data
(63) Continuation of application No. PCT/EP2023/062464, filed on May 10, 2023.

(30) Foreign Application Priority Data
May 11, 2022 (EP) 22172801.7

(51) Int. Cl. G01N 33/574; G01N 33/74

(52) U.S. Cl. CPC G01N 33/57488; G01N 33/57492; G01N 33/74; G01N 2333/62

(57) ABSTRACT

The present invention relates to the use of BMMF Rep-protein as diagnostic marker for pancreatic cancer and diabetes.

### CROSS-REFERENCE TO RELATED APPLICATIONS

This application is a continuation of PCT/EP2023/062464, filed May 10, 2023; which claims the benefit of European Application No. 22172801.7, filed May 11, 2022. The contents of the above-identified applications are incorporated herein by reference in their entireties.

### SEQUENCE LISTING

This application contains an ST.26 compliant Sequence Listing, which was submitted in xml format via Patent Center and is hereby incorporated by reference in its entirety. The .xml copy, created on May 11, 2023 is named “2023 May 11 sequence listing.xml” and is 30000 bytes in size

### TECHNICAL FIELD

The invention relates to the use of a DNA-replication-associated (Rep) protein as a diagnostic marker for pancreatic cancer and diabetes mellitus type 2.

### BACKGROUND OF THE INVENTION

The pancreas serves as an exocrine gland that secretes pancreatic juice which is transported to the digestive tract through the pancreatic duct, while also functioning as an endocrine gland that secretes hormones such as insulin and glucagon into blood. Thus, diseases of the pancreas are often a cause of or are closely connected with diabetes.

Since the pancreas is surrounded by many organs such as the stomach, the duodenum, the small intestine, the liver, and the gallbladder, pancreatic cancer is not only difficult to detect early but has properties such as a lack of subjective symptoms, very rapid progression, and metastasis to other organs and thus has very poor prognosis as compared with other cancers. Despite several new cancer treatment options, e.g. immunotherapeutic approaches with checkpoint inhibitors or bispecific antibodies, the 5-year relative survival rates by cancer types remain lowest with between 6% and 10% in pancreatic cancer

The basic therapy of pancreatic cancer is practiced by surgery, systemic chemotherapy, radiotherapy, or a combination thereof depending on a stage of progression. Although 15 to 20% pancreatic cancer patients undergo surgery for potential complete cure, the great majority of patients will not benefit from surgery due to local progression or metastasis.

The UICC (Unio Internationalis Contra Cancrum) stages of progression of pancreatic cancer are classified into stages 0, IA, IB, IIA, IIB, III, IVa, and IVb. Stages I to III occupy half or more of the number of 5-year survivals, and stages IVa and IVb occupy 70% or more of the stages of progression at the time of diagnosis. Therefore, early detection and treatment of pancreatic cancer are necessary.

Abdominal ultrasonography is a very useful, convenient and minimally invasive examination method in check-ups for patient care or medical examination. However, it is often difficult to visualize pancreatic cancer having a small tumor size or a lesion on the pancreatic tail side. In medical check-ups, the abnormality detection rate with pancreatic images by abdominal ultrasonography is approximately 1%, and the detection rate of pancreatic cancer is approximately 0.05% or lower.

Meanwhile, cystic diseases that occur in the pancreas are known to progress to invasive cancers through malignant transformation and can be regarded as pancreatic cancer precursor lesions. The malignancy of the cystic diseases is evaluated on the basis of cyst diameters, wall thickening, diameters of the main pancreatic duct, mural nodules, stenosis of the main pancreatic duct, enlarged lymph nodes, and cystic lesions, etc. Patients with mucinous cystic tumors (also called mucinous cystadenom), one type of benign cystic disease, have a high probability for developing malignant cancer and are therefore recommended to receive regular follow-ups or tumor resection, even if there is no malignancy of the neoplasms when they are detected.

To enhance earlier detection, there is a need for biomarkers that will facilitate early detection and further insights into the pathogenesis of pancreatic cancer.

### DESCRIPTION OF THE PRESENT INVENTION

The inventors found that the uptake of BMMF (Bovine Meat and Milk Factor) agents within the first months of life either by substitution of breast-feeding during weaning by cow milk products or by the uptake of dairy or beef products, in general, leads to the early infection of newborns with BMMF antigens. The early exposure is thereby hypothesized to induce an immunological state in which the individuals will not clear or neutralize BMMF which is taken up via the nutrition in the following part of life as efficiently as individuals which profited from a longer breastfeeding period. Within the next years or decades-depending on the immune system of the host additional BMMF antigens accumulate within the stroma of pancreatic tissue. This accumulation may be triggered also by the uptake of specific molecules that represent receptors for BMMFs. These molecules are also taken up by consumption of cow products and are incorporated into lectin receptors on the surface of the host cells. When a certain level of antigen is reached by continuous uptake of BMMFs in combination with focal spreading of infection, the host immune response induces a state of chronic and local inflammation producing a stable increase of diffusing, reactive oxygen species (ROS) and cyclooxygenase-2 (Cox-2) which dramatically increases the probability of deregulated cell proliferation with concomitant fixation of random mutations in surrounding cells induced by ROS (de Villiers and zur Hausen, 2021). Especially, cells with intrinsically high replicative activity might represent targets enriching random DNA mutations enabling stochastic manifestation of mutations as a basic requirement for tumorigenesis and development of pancreatic cancer. Thus, BMMFs represent a specific and local trigger for induction of chronic inflammation within the tissue stroma leading to an increase of ROS which induces proliferation and mutation in surrounding replicative cells eventually leading to the formation of hyperplasia as precursors for cancer.

In detail, structural properties of pleomorphic vesicles, regularly identified by staining tissues of prostate cancer patients for expression of Rep protein of a subgroup of BMMF1, were observed. The latter represent proteins involved in replication of small single-stranded circular plasmids of BMMF, but most likely also contributing to pleomorphic vesicular structures found in the periphery of pancreatic cancers. Gold-labelled, structurally dense regions are demonstrated in preselected areas of pancreatic cancer, after staining with gold-labelled monoclonal antibodies against BMMF1 Rep. Similar structures were observed in human embryonic cells (HEK293TT) overexpressing Rep. These data suggest that Rep or Rep isoforms contribute to the structural formation of vesicles. Additionally, a co-localization of the anti-Rep stained signals with CD68-positive macrophages was observed. The regions with highest Rep-specific antibody detection correlate with regions with highest detection levels for CD68 positive cells pointing towards a localization of the Rep-specific antigens in inflammatory tissue areas, i.e. regions with especially high levels of inflammatory monocytes, circulating macrophages, or resident tissue macrophages.

Bovine sera were initially subjected to density gradient centrifugation. Single fractions of these gradients were analysed for structural particles by electron microscopy, as well as DNA extraction, amplification and sequencing. Comparison to all databanks revealed a DNA sequence homology to the transmissible spongiform encephalopathy (TSE)-associated isolates Sphinx 1.76 (1,758 bp; accession no. HQ444404) and Sphinx 2.36 (2,360 bp; acc no. HQ444405) (Manuelidis L. 2011). Full-length plasmid-like, episomal DNA genomes of Bovine Meat and Milk Factors (BMMF) were obtained using abutting primers and PCR amplification on the partial DNA fragments. The main open reading frame of all isolates was identified in silico as a putative replication gene (Rep). and revealed a remarkable degree of similarity to specific plasmids of Acinetobacter baumannii and Psychrobacter species. More than 130 BMMF DNA genomes were subsequently isolated from milk and other dairy products, as well as 3 isolates from a brain biopsy and serum from multiple sclerosis patients. These isolates were grouped as BMMF1, BMMF2 and BMMF4 based on DNA sequence homology (Funk et al. 2014, Gunst et al. 2014, Whitley et al. 2014, Falida et al., 2017, de Villiers et al., 2019). Detailed analyses of their genomes revealed characteristics of both viruses and bacterial plasmids needed for replication, transcription and translation, frequently all present in a single genome, thereby constituting a novel class of pathogens (de Villiers et al. 2019).

Two BMMF1 isolates from a brain biopsy of a MS patient were MSBI1.176 (MSBI, multiple sclerosis brain isolate) (1,766 bp) and MSBI2.176 (1,766 bp) which are designated as “MSBI1 genome” and “MSBI2 genome”, respectively. MSBI1.176 shares 98% nucleotide similarity to the sequence of Sphinx 1.76. The sequences of the isolates MSBI1.176 and MSBI2.176 have been deposited in the EMBL Databank under accession numbers LK931491 (MSBI1.176) and LK931492 (MSBI2.176) (Whitley C. et al. 2014) and have been aligned and described in WO 2016/005054 A2.

Further isolates were obtained from cow milk, dairy products and bovine sera. These Cow milk isolates (CMI) were CMI1.252, CMI2.214, CMI3.168, CMI4.158, HCBI3.108, HCB14.296, HCBI6.252, CMI.M3.1 and CMI.9M.1 which are designated as “CMI1 genome”, “CMI2 genome”, “CMI3 genome”, “CMI4 genome”, “HCBI3 genome”, “HCBI4 genome”, “HCB16 genome”, “C1MI.3M.1 genome”, “C1MI.9M.1 genome”, respectively. The sequences of the isolates have been deposited in the EMBL Databank under accession numbers LK931487 (CMI1.252), LK931488 (CMI2.214), LK931489 (CMI3.168), LK931490 (CMI4.158), LK931495 (HCBI3.108), LK931496 (HCBI4.296), LK931493 (HCBI6.252), LR215499 (C1MI.3M.1), LR215496 (C1MI.9M.1) and have been aligned and described in part in WO 2016/005054 A2. The CMI5 genomes are described in Falida et al., 2017.

The present inventors have found that both CMI genomes and MSBI genomes replicate and show a significant production of transcribed RNA in vitro. Antibodies against the MSBI1.176 Rep show expression in the peripheral tissue around the pancreatic cancer tissue. The present inventors have found that the encoded Rep proteins of a subgroup of BMMF1 infectious agents (MSBI1 Rep, MSBI2 Rep, CMI1 Rep, CMI2 Rep, CMI3 Rep, CMI4 Rep, HCBI3 Rep, HCBI4 Rep, HCB16 Rep, C1MI.3M.1 Rep, C1MI.9M.1 Rep) represent a diagnostic marker for pancreatic cancer.

The inventors have raised monoclonal antibodies against Rep protein using full-length MSBI1.176 Rep or consensus peptides derived from two conserved regions of the MSBI1.176 Rep sequence. In particular embodiments the anti-Rep antibodies bind to epitopes of Rep protein that are exemplified in FIG. 5. Particular preferred antibodies bind to epitopes within an amino acid sequence selected from the group consisting of amino acids from 1 to 136, from 137 to 229 and from 230 to 324 of SEQ ID NO:1. For example, the antibody binds to an epitope comprised by SEQ ID NO:2 or SEQ ID NO:3.

### BRIEF DESCRIPTION OF THE FIGURES

FIG. 1 IHC detection of BMMF1 Rep on PDAC cancer patient tissue in consecutive tissue sections in CD68-enriched tissue regions (A) and in insulin-positive beta cells of Langerhans islands (B). Ctrl=Control

FIG. 2 Ultrastructural representation of BMMF in tissues of a pancreatic cancer patient.

(A) IHC DAB staining of tissue peritumor pancreatic cancer tissue stained with anti-Rep AB3. Both endocrine cells (I, beta cells in Langerhans islets) and cells in the exocrine tissue region (II) are stained. (B) Pre-IEM of a pancreatic cancer FFPE biopsy of the same donor: Exocrine (upper row) and endocrine (low row) regions of peritumor pancreatic cancer tissue stained with AB3 showing gold-decorated pleomorphic structures.

FIG. 3 Ultrastructural representation of BMMF in tissues a pancreatic cancer patient stained with gold-labelled anti-Rep AB3 after pre-embedding immuno-electron microscopy (pre IEM)

BMMF pleomorphic vesicles in human FFPE biopsies of exocrine and endocrine peritumor pancreatic cancer tissue regions. The tissues exhibit small, approximately 100 nm-sized pleomorphic bodies of a structurally-dense material that heavily stains with gold-labelled BMMF antibody. Even though no structural motif was identified, filamentous arrangements at sites indicate higher-order organization (arrowheads). Size of gold grains: 10 nm.

FIG. 4 MSBI1.176 Rep overexpression in HEK293TT analyzed by western blotting.

3xFlag-H1MSB.1 Rep (expected molecular size of 40 kDa, arrowhead) or a non-tagged Rep (37 kDa, arrowhead) were overexpressed in HEK293TT for 48 h and analyzed by SDS-PAGE and western blotting with anti-Rep (AB3 and AB7) and anti-Flag antibodies. Mock control transfection was performed with pcDNA3.1(-) vector without Rep gene insert and GAPDH served as loading control. (B) PonceauS protein staining of the respective membranes.

FIG. 5 Ultrastructural demonstration by cryo-IEM of BMMF expression in HEK293TT.

Cells were transfected with either a plasmid for expression of H1MSB.1 Rep, 3xFlag-H1MSB.1 Rep, or the empty vector for control. Rep overexpression leads to formation of large segregation structures (asterisks) which stain intense with anti-Rep AB3 and AB7 and, in case of overexpressed 3xFlag-Rep, also with anti-Flag. These structures did not stain with the isotype control antibody. No such structures formed in cells transfected with the empty vector. Size of gold grains: 10 nm; M: mitochondrion.

FIG. 6 Membrane-associated BMMF in HEK293TT cells after overexpression of MSBI1.176 Rep.

HEK293TT cells transiently transfected with a MSBI1.176 Rep expression plasmid (untagged, codon optimized) express partitioned pleiomorphic segregation structures which stain densely with AB7 (cryo IEM, A and B), as well as (C) with uranyl and lead contrasting agents for conventional resin TEM. Segregation structures typically occupy the cytoplasm of cells (1) and occasionally also the nucleoplasm (2). In addition, similar material accumulate along cellular membranes (C enlarged views to the right, asterisks). Size of gold grains: 10 nm; N: nucleus, M: mitochondrion.

FIG. 7 Modell conception of the etiology of pancreatic cancer and diabetes mellitus type 2 and their mutual relationship to each other

FIG. 8 Localisation of the AB epitopes within MSBI1.176 Rep

FIG. 9 Quantification of Rep, CD68, and CD163 immunofluorescence microscopic staining

Semi-automated, cell-based quantification of Rep, CD68, and CD163 immunofluorescence microscopic staining of individual FFPE tissues from tumour (PDAC, n=20) and pathologists-validated tumour-adjacent tissues of pancreatic ductal adenocarcinoma patients (n=6) and from non-cancer individuals (healthy donors, n=17) as well as tissues from chronic pancreatitis patients (CP, n=6). Mean representation of Rep+CD68+cells in the respective tissues. Test with one-way ANOVA, Tukey's multiple comparisons. Significance: *P<0.05, **P<0.01, ***P<0.001 ****P<0.0001

FIG. 10 Quantification of Rep+CD68+CD163+ cells of individual FFPE tissues

Semi-automated, cell-based quantification of Rep+CD68+CD163+ cells of individual FFPE tissues from tumour (PDAC, n=20) and pathologists-validated tumour-adjacent tissues of pancreatic ductal adenocarcinome patients (adjacent to PDAC, n=6) and from non-cancer individuals (Ctr, n=17) as well as tissues from chronic pancreatitis patients (CP, n=6). Mean representation of Rep+CD68+CD163+ cells within all macrophage cells in the respective tissues. Test with one-way ANOVA, Tukey's multiple comparisons. Significance: *P<0.05, **P<0.01, ***P<0.001 ****P<0.0001

FIG. 11 Immunohistochemical staining

Immunohistochemical staining of representative FFPE tissue of PDAC, chronic pancreatitis and healthy donors based on anti-Rep AB10-3 in (staining with diaminobenzidine (DAB), scale bar: 50 μm).

FIG. 12 Semi-automated quantification of Rep-positive area and Rep staining intensity

Semi-automated quantification of Rep-positive area (%) and Rep staining intensity (a.u.) in Langerhans islets of healthy donors (Ctr, n=17), chronic pancreatitis patients (CP, n=6) and pancreatic ductal adenocarcinoma patients (PDAC, n=20) (representation of mean). Tested by on-way ANOVA, Tukey's multiple comparisons. Significance: *P<0.05, ****P<0.0001

The invention provides the teaching that Rep proteins may represent diagnostic markers for an enhanced risk to develop pancreatic cancer or to diagnose pancreatic cancer or to confirm a pancreatic cancer diagnosis.

The terms “pancreatic cancer” means a cancer that evolved as a consequence of uncontrolled cell growth in the pancreatic gland. These malignancies may develop as a consequence of pre-existing benign cysts and hyperplasias where genetic alterations promote the transition from normal to cancerous growth. The term “pancreatic cancer” means pre-stages, early stages or late stages of the disease and metastases derived therefrom. “Pancreatic cancer” embraces all malignant neoplasms of the pancreas head, pancreas body, pancreas tail or of the Ductus pancreaticus. It may concern the exocrine or endocrine cells and embraces all types of exocrinic or endocrinic neoplasms. The most common exocrinic tumors are pancreatic ductal adenocarcinoma (PDAC), tumors of the Papilla Vateri, intraductal papillary-mucinous tumor (IPMT) or acinus cell carcinoma. The most common endocrinic tumors are insulinoma, gastinoma, somatostatinoma or glucagenoma.

In an alternative embodiment the present invention may also encompass the systematic testing of healthy pancreatic tissue (tissue from individuals without cancer diagnosis or a specific hint for the disease) to assess the disease risk in the future. This means that the present invention is also suitable to determine the predisposition for developing pancreatic cancer by use of the BMMF Rep as an early diagnostic marker.

“Predisposition” means, according to the accepted meaning in medicine, the tendency to a medicinal condition. It is the medicinal susceptibility to a specific disease or a condition of special susceptibility to a disease, usually based on the combined effects of genetic and environmental factors.

“Rep protein” as used herein refers to a DNA-replication-associated protein. The Rep protein comprises DNA binding activity and could be essential for initiation of replication of episomal/viral DNA molecules. In general Rep protein refers to a Rep protein from the group of BMMF1 proteins (de Villiers et al., 2019). In particular, the Rep protein is a MSBI1 genome-encoded Rep protein (MSBI1 Rep), a MSBI2 genome-encoded Rep protein (MSBI2 Rep), a CMI1 genome-encoded Rep protein (CMI1 Rep), a CMI2 genome-encoded Rep protein (CMI2 Rep), CMI3 genome-encoded Rep protein (CMI3 Rep), CMI4 genome-encoded Rep protein (CMI4 Rep), HCBI4 genome-encoded Rep protein (HCBI4 Rep), HCBI6-encoded Rep protein (HCB16 Rep), C1MI.3M.1 genome-encoded Rep protein (C1MI.3M.1 Rep), C1MI.9M.1 genome-encoded protein (C1MI.9M.1 Rep). Preferably, the MSBI1 Rep protein is encoded by MSBI1.176 deposited in the EMBL databank under the acc. no. LK931491 and has the amino acid sequence as depicted in SEQ ID NO:1 or the Rep protein is MSBI2 encoded by MSBI2.176 deposited in the EMBL databank under the acc. no. LK931492 and has the amino acid sequence as depicted in SEQ ID NO:8 (Whitley, Gunst et al. 2014). In another preferred embodiment the CMI1 Rep protein is encoded by CMI1.252 deposited in the EMBL databank under the acc. no. LK931487 and has the amino acid sequence as depicted in SEQ ID NO:10. In another preferred embodiment the CMI2 Rep protein is encoded by CMI2.214 deposited in the EMBL databank under the acc. no. LK931488 and has the amino acid sequence as depicted in SEQ ID NO:11. In another preferred embodiment the CMI3 Rep protein is encoded by CMI3.168 deposited in the EMBL databank under the acc. no. LK931489 and has the amino acid sequence as depicted in SEQ ID NO:12. In another preferred embodiment the CMI4 Rep protein is encoded by CMI4.158 deposited in the EMBL databank under the acc. no. LK931490 and has the amino acid sequence as depicted in SEQ ID NO:16. In another preferred embodiment the HCBI3 is encoded by HCBI3.108 deposited in the EMBL databank under the acc. no. LK931495 and has the amino acid sequence as depicted in SEQ ID NO:17. In another preferred embodiment the HCBI4 is encoded by HCB14.296 deposited in the EMBL databank under the acc. no. LK931496 and has the amino acid sequence as depicted in SEQ ID NO:18. In another preferred embodiment the C1MI.3M.1 is deposited in the EMBL databank under the acc. no. LR215499 and has the amino acid sequence as depicted in SEQ ID NO:19. In another preferred embodiment the C1MI.9M.1 is deposited in the EMBL databank under the acc. no. LR215496 and has the amino acid sequence as depicted in SEQ ID NO:20.

In a particular preferred embodiment the Rep protein comprises a N-terminal region conserved among BMMF1 genomes consisting essentially of amino acids from 1 to 229 of SEQ ID NO:1 and a C-terminal variable region specific for MSBI1.176 consisting essentially from amino acids 230 to 324 of SEQ ID NO:1. The N-terminal conserved region comprises a putative, first DNA binding domain consisting essentially of amino acids from 1 to 136 of SEQ ID NO:1 and a second putative DNA binding domain consisting essentially of amino acids from 137 to 229 of SEQ ID NO:1. The C-terminal domain shows little sequence homology with any known protein and consists of amino acids 230 to 324.

“Rep protein” also encompasses fragments and variants of the protein with SEQ ID NO:1, SEQ ID NO:8, SEQ ID. NO:10, SEQ ID. NO: 11, SEQ ID. NO:12, SEQ ID. NO:16, SEQ ID. NO:17, SEQ ID NO:18, SEQ ID NO:19 and/or SEQ ID. NO:20 which are capable of binding an anti-Rep antibody specific for Rep protein having the amino acid sequence of SEQ ID NO:1. Preferably, such a fragment is an immunogenic fragment of the protein having the amino acid sequence of SEQ ID NO:1, SEQ ID NO:8, SEQ ID. NO:10, SEQ ID. NO: 11, SEQ ID. NO:12, SEQ ID. NO:16, SEQ ID. NO:17, SEQ ID NO:18, SEQ ID NO:19 and/or SEQ ID. NO:20 which encompasses at least one epitope for an anti-Rep protein antibody against the Rep protein of SEQ ID NO:1. and, preferably, comprises at least 7, 8, 9, 10, 15, 20, 25 or 50 contiguous amino acids. In particular embodiments the fragment comprises or consists essentially of a domain of the Rep protein, for example, the N-terminal conserved region, the C-terminal variable region, the first or second DNA binding domain. A variant of the protein with SEQ ID NO:1, SEQ ID NO:8, SEQ ID. NO:10, SEQ ID. NO: 11, SEQ ID. NO:12, SEQ ID. NO:16, SEQ ID. NO:17, SEQ ID NO:18, SEQ ID NO:19 and/or SEQ ID. NO:20 comprises one or more amino acid deletions, substitutions or additions compared to respective sequence and has a homology of at least 90%, 91%, 92%, 93%, 94%, 95%, 96%, 97%, 98% or 99% to the amino acid sequence of SEQ ID NO:1, SEQ ID NO:8, SEQ ID. NO:10, SEQ ID. NO: 11, SEQ ID. NO:12, SEQ ID. NO:16, SEQ ID. NO:17, SEQ ID NO:18, SEQ ID NO:19 and/or SEQ ID. NO:20, wherein the variant is capable of binding an anti-Rep antibody specific for a Rep protein having the amino acid sequence of SEQ ID NO:1. Included within the definition of variant are, for example, polypeptides containing one or more analogues of an amino acid (including, for example, unnatural amino acids, peptide nucleic acid (PNA), etc.), polypeptides with substituted linkages, as well as other modifications known in the art, both naturally occurring and non-naturally occurring. The term Rep protein includes fusion proteins with a heterologous amino acid sequence, with a leader sequence or with a Tag-sequence and the like. In certain embodiments of the invention protein tags are genetically grafted onto the Rep protein described above, for example the Rep protein selected from the group consisting of MSBI1, MSBI2, CMI1, CMI2, CMI3, CMI4, HCBI4, HCBI6, C1MI.3M.1 or C1MI.9M.1. In particular at least one protein tag is attached to a polypeptide consisting of an amino acid sequence as depicted in any one of SEQ ID NOs: 1-3, 8-12, 14 and 16-20. Such protein tags may be removable by chemical agents or by enzymatic means. Examples of protein tags are affinity or chromatography tags for purification. For example, the Rep protein may be fused to a Tag-sequence, for example, selected from the group consisting of His₆-Tag (SEQ ID NO:4), T7-Tag (SEQ ID NO:5), FLAG-Tag (SEQ ID NO:6) and Strep-II-Tag (SEQ ID NO:7). a His-Tag (SEQ ID No:4), a T7-Tag (SEQ ID NO:5), FLAG-Tag (SEQ ID NO:6) or StrepII-Tag (SEQ ID NO:7). Further, fluorescence tags such as green fluorescence protein (GFP) or its variants may be attached to a Rep-protein according to the invention.

In a particular preferred embodiment the MSBI1 genome-encoded Rep protein (MSBI1 Rep) is codon-optimized for the production in human cell lines (e.g. HEK-293, HEK293TT, HEK293T, HEK293FT, HaCaT, HeLa, SiHa, CaSki, HDMEC, L1236, L428, BJAB, MCF7, Colo678) as well as bovine (e.g. MAC-T) or murine cell lines (e.g. GT1-7) or any other cell line. This is described in detail in PCT/EP2017/075774.

The Rep protein of the invention, including the Rep fragments and Rep variants as defined above, can be prepared by classical chemical synthesis. The synthesis can be carried out in homogeneous solution or in solid phase. The polypeptides according to this invention can also be prepared by means of recombinant DNA techniques. “Subject” as used herein refers to a mammalian individual or patient, including murines, cattle, for example different bovine species, simians and humans. Preferably, the subject is a human patient.

“Anti-Rep antibody” as used herein refers to an antibody binding at a detectable level to Rep protein which affinity is more strongly to the Rep protein of the invention than to a non-Rep protein. Preferably, the antigen affinity for Rep protein is at least 2-fold, more preferably 5-fold or 10-fold, larger than background binding. In particular, the anti-Rep antibody detects the MSBI1 Rep having the amino acid sequence of SEQ ID NO:1. In particular embodiments the antibody detects MSBI1 Rep, MSBI2 Rep, CMI1 Rep, CMI2 Rep, CMI3 Rep, CMI4 Rep, HCBI4 Rep, HCBI6 Rep, C1MI.3M.1 Rep, and/or C1MI.9M.1 Rep. In certain embodiments the anti-Rep antibody detects at least two, preferably all, of MSBI1 Rep, MSBI2 Rep, CMI1 Rep, CMI2 Rep, CMI3 Rep, CMI4 Rep HCBI4 Rep, HCB16 Rep, C1MI.3M.1 Rep, and/or C1MI.9M.1 Rep.

The inventors also tested the antibody level of pancreatic cancer patients by contacting the Rep protein with a specimen suspected of containing anti-Rep protein antibodies under conditions that permit the Rep protein to bind to any such antibody present in the specimen. Such conditions will typically be physiologic temperature, pH and ionic strength using an excess of Rep protein. The incubation of the Rep protein with the specimen is followed by detection of immune complexes comprised of the antigen. In certain embodiments either the Rep protein is coupled to a signal generating compound, e.g. detectable label, or an additional binding agent, e.g. secondary anti-human antibody, coupled to a signal generating compound is used for detecting the immune complex.

Anti-Rep antibodies can be detected and quantified in assays based on Rep protein as protein antigen, which serves as target for the mammalian, e.g. human, antibodies suspected in the specimen. Preferably, the Rep protein is purified and the specimen can be, for example, serum or plasma. The methods include immobilization of Rep protein on a matrix followed by incubation of the immobilized Rep protein with the specimen. Finally, the Rep-bound antibodies of the formed immunological complex between Rep protein and antibodies of the specimen are quantified by a detection binding agent coupled to a signal generating compound, e.g. secondary HRP-(horseradish-peroxidase)-coupled detection antibody allowing for HRP-substrate based quantification. This signal generating compound or label is in itself detectable or may be reacted with an additional compound to generate a detectable product.

Design of the immunoassay is subject to a great deal of variation, and many formats are known in the art. Protocols may, for example, use solid supports, or immunoprecipitation. Most assays involve the use of binding agents coupled to signal generating compounds, for example labelled antibody or labelled Rep protein; the labels may be, for example, enzymatic, fluorescent, chemiluminescent, radioactive, or dye molecules. Assays which amplify the signals from the immune complex are also known; examples of which are assays which utilize biotin and avidin or streptavidin, and enzyme-labelled and mediated immunoassays, such as ELISA assays.

The immunoassay may be in a heterogeneous or in a homogeneous format, and of a standard or competitive type. Both standard and competitive formats are known in the art.

In an immunoprecipitation or agglutination assay format the reaction between the Rep protein and the anti-Rep antibody forms a network that precipitates from the solution or suspension and forms a visible layer or film of precipitate. If no anti-Rep antibody is present in the specimen, no visible precipitate is formed.

In further embodiments the inventors used methods wherein an increased amount of Rep protein in a sample correlates with a diagnosis or predisposition of pancreatic cancer. In such embodiments the Rep protein in the sample is detected by anti-Rep antibodies.

“Sample” as used herein refers to a biological sample encompassing cancerous pancreatic tissue, peripheral tissue surrounding the cancerous tissue and (benign) hyperplasias. The samples encompass tissue samples such as tissue cultures or biopsy specimen.

Such methods (ex-vivo or in-vitro) comprise the steps of detecting Rep protein in a sample from a subject by anti-Rep antibodies. In such methods Rep protein is detected in tissue samples by immunohistochemical methods or immunofluorescence microscopy.

In certain embodiments anti-Rep antibodies are used for the detection or capturing of the Rep protein in the sample.

The term “antibody”, preferably, relates to antibodies which consist essentially of pooled polyclonal antibodies with different epitopic specificities, as well as distinct monoclonal antibody preparations. As used herein, the term “antibody” (Ab) or “monoclonal antibody” (Mab) is meant to include intact immunoglobulin molecules as well as antibody fragments (such as, for example, Fab and F(ab′)2 fragments) which are capable of specifically binding to Rep protein. Fab and F(ab′)2 fragments lack the Fc fragment of intact antibody, clear more rapidly from the circulation, and may have less non-specific tissue binding than an intact antibody. Thus, these fragments are preferred, as well as the products of a FAB or other immunoglobulin expression library. Moreover, antibodies useful for the purposes of the present invention include chimeric, single chain, multifunctional (e.g. bispecific) and humanized antibodies or human antibodies.

In certain embodiments the antibody or antigen binding fragment thereof is coupled to a signal generating compound, e.g., carries a detectable label. The antibody or antigen binding fragment thereof can be directly or indirectly detectably labelled, for example, with a radioisotope, a fluorescent compound, a bioluminescent compound, a chemiluminescent compound, a metal chelator or an enzyme. Those of ordinary skill in the art will know of other suitable labels for binding to the antibody, or will be able to ascertain such, using routine experimentation.

Anti-Rep antibodies are, preferably, raised (generated) against a Rep protein having the amino acid sequence of SEQ ID NO:1 or a fragment thereof by methods well known to those skilled in the art.

In certain embodiments anti-Rep antibodies are used in the methods of the invention which are capable of binding to several or all kinds of Rep proteins from the group of the BMMF1 proteins. Such antibody binds to an epitope within the conserved N-terminal region of the Rep protein from amino acids 1 to 229 of SEQ ID NO:1. In particular embodiments anti-Rep antibodies of the anti-BMMF1 Rep type are used which bind to an epitope within SEQ ID NO:2 (amino acids 32-49 of SEQ ID NO:1) or SEQ ID NO:3 (amino acids 197-216 of SEQ ID NO:1). The peptide fragments of SEQ ID NO:2 and SEQ ID NO:3 are highly conserved among the Rep proteins from BMMF1 and appear to be exposed due to their hydrophilic character. Anti-Rep antibodies of this anti-BMMF1 type may be produced by immunization, for example of mice or guinea pig, by peptides consisting essentially of the amino acid sequences as depicted in SEQ ID NOs: 2 or 3; or by other immunogenic fragments, preferably comprising at least 8-15 amino acids, derived from the conserved N-terminal Rep protein region from amino acids 1 to 229 of SEQ ID NO:1.

In further embodiments anti-Rep antibodies specific for MSBI1 Rep protein are used. Such antibodies may be produced, for example, by immunization of a mammal such as mice or guinea pig with a full-length Rep protein having the amino acid sequence of SEQ ID NO:1.

Preferably, the methods of the invention use anti-Rep antibodies which are capable of detecting Rep protein up to ranges from pictogram to femtogram.

Examples of such groups of anti-Rep antibodies are shown in Table 1:

| Antibody | Rep-Protein | | | DSMZ |
|---|---|---|---|---|
| Group | Localisation | Detection | Antibody | deposit |
| Group A | cytoplasm + | BMMF1 | AB01 523-1-1 | DSM |
| | nuclear membrane | Reps | (Ab 1-5) | ACC3327 |
| | (+nucleus) | | | |
| Group B | speckles in | BMMF1 | AB02 304-4-1 | DSM |
| | cytoplasm | Reps | (Ab 5-2) | ACC3328 |
| Group C | cytoplasm + | MSBI1 | MSBI1 381-6-2 | DSM |
| | nuclear membrane | Rep | (Ab 3-6) | ACC3329 |
| | (+nucleus) | | MSBI1 572-13- | |
| | | | 19 (Ab 10-3; | |
| | | | AB10) | |
| | | | MSBI1 617-1-3 | |
| | | | (Ab 11-5) | |
| Group D | speckles in | MSBI1 | D1: MSBI1 | DSM |
| | cytoplasm | Rep | 961-2-2 (Ab 9-2) | ACC3331 |
| | | | D2: MSBI1 | DSM |
| | | | 761-5-1 (Ab 13) | ACC3330 |

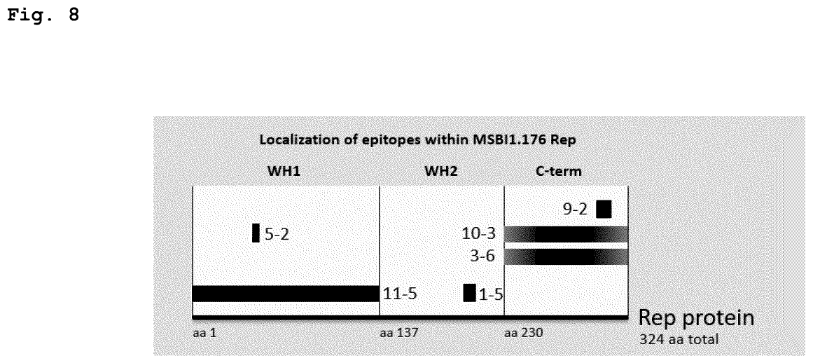

The localization of the antibody epitopes within MSBI1.176 Rep is shown in FIG. 8.

Anti-Rep antibodies of group A have an epitope within the amino acid sequence depicted in SEQ ID NO:3 (aa 198-217 of SEQ ID NO:1) and are capable of detecting MSBI1 Rep and Rep proteins comprising this conserved epitope of the BMMF1 group (e.g. MSBI2, CMI1, CMI2, CMI3, CMI4, HCBI4, HCBI6, C1MI.3M.1, C1MI.9M.1). In immunofluoresence assays such anti-Rep antibodies detect a specific Rep localisation pattern, wherein the main localisation is homogeneously distributed over the cytoplasm and nuclear membrane; and additional weak and homogeneously distributed localisation is seen in the nucleus. An example of such a group A antibody is antibody AB01 523-1-1 (also called antibody 1-5; DSM ACC3327) which was employed in the examples as group A antibody.

Anti-Rep antibodies of group B have an epitope within the amino acid sequence depicted in SEQ ID NO:2 (aa 33-50 of SEQ ID NO:1) and are capable of detecting MSBI1 Rep and Rep proteins comprising this conserved epitope of the BMMF1 group (e.g. MSBI2, CMI1, CMI2, CMI3, CMI4, HCBI4, HCBI6, C1MI.3M.1, C1MI.9M.1). In immunofluoresence assays such anti-Rep antibodies detect specifically speckles (cytoplasmatic aggregations) of the Rep protein (often in the periphery of the nuclear membrane). An example of such a group B antibody is the antibody designated as AB02 304-4-1 (also called antibody 5-2; DSM ACC3328) which was employed in the examples as group B antibody.

Anti-Rep antibodies of group C detect specifically a structural epitope of MSBI1 (SEQ ID NO:1). In immunofluoresence assays such anti-Rep antibodies detect a specific Rep localisation pattern, wherein the main localisation is homogeneously distributed over the cytoplasm and nuclear membrane; and additional weak and homogeneously distributed localisation is seen in the nucleus. An example of such a group C antibody is antibody MSBI1 381-6-2 (also called antibody 3-6 or antibody 3; DSM ACC3329) which was employed in the Example as group C antibody with an epitope in the sequence of aa 230-324. Another example of an antibody of a group C antibody is antibody MBSI1 572-13-19 (also called antibody 10-3 or antibody 10) detecting an epitope in the C-terminal domain of MSBI 1 Rep (aa 230-324). Another example of an antibody of a group C antibody is antibody MBSI1 617-1-3 (also called antibody 11-5) detecting an epitope in the N-terminal domain of MSBI 1 Rep (aa 1-136).

Anti-Rep antibodies of group D detect specifically a structural epitope of MSBI1 (SEQ ID NO:1), where antibody MSBI1 961-2-2 designated as “D1” (also called antibody 9-2; DSM ACC3331) detects an epitope depicted in SEQ ID NO:9 (aa 281-287) in the C-terminal domain of MSBI1. Antibody MSBI1 761-5-1 (also called antibody 13; DSM ACC3328) designated as “D2” detects a 3D structural epitope of MSBI1 which is exclusively accessible under in vivo conditions and is not accessible in Western Blots. In immunofluoresence assays such anti-Rep antibodies detect specifically speckles (cytoplasmatic aggregations) of the Rep protein (often in the periphery of the nuclear membrane.

The availability of BMMF-specific Rep monoclonal antibodies and the demonstration of the DNA of these infectious agents in peritumour tissues, prompted the inventors to investigate patient tissue for the presence of specific structural components by immunogold electromicroscopy using primary Rep monoclonal antibodies as tool. The identification of such gold-labelled structures in peritumor tissues of a pancreatic cancer patient is described in detail in the following examples.

The present ultrastructural analyses by immuno-electron microscopy (IEM) demonstrates BMMF Rep expression in macrophages in peritumor pancreatic cancer tissues. The gold decorated targets show a pleomorphic appearance, differing in size and shape and separate from the surrounding area. In the peritumor of pancreatic cancer, pleomorphic structures with diameters approximately between 50-200 nm were found. Cryo IEM as well as conventional resin TEM, which both allow for more delicate structural preservation compared to pre-embedding IEM of FFPE (formalin-fixed paraffin-embedded tissue) samples, show MSBI1.176 Rep overexpression-dependent segregation bodies in the cytoplasm of HEK293TT cells with potential to associate with membranes. In all modes of sample preparation, the detailed view into focal accumulations reveal ultrafine structures like voids and filaments indicative for higher order organization.

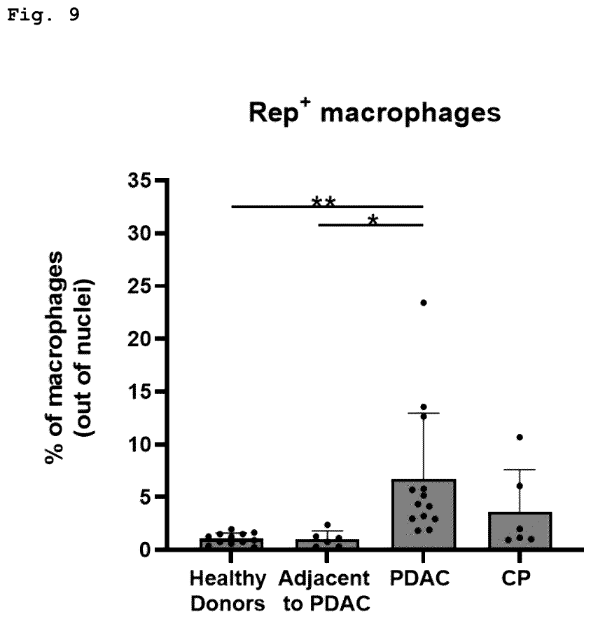

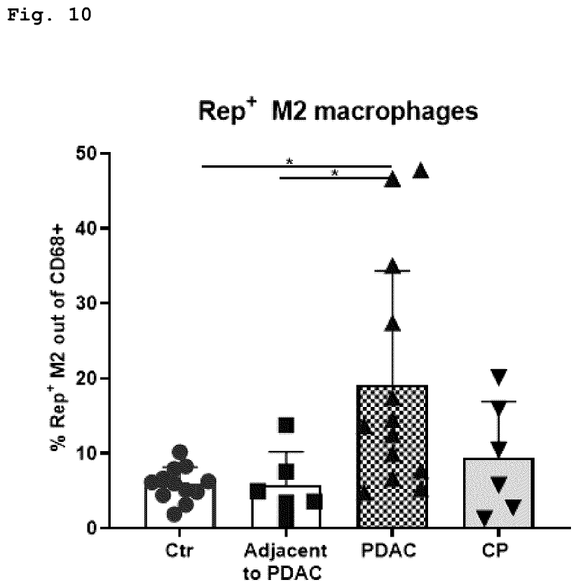

Semi-automated quantification of Rep, CD68, and CD163 immunofluorescence microscopic staining of individual FFPE tissues was used to compare the level Rep+, Rep+CD68+, Rep+CD68+CD163+ and CD68+ cells in the tissues of pancreatic cancer tumour and pathologist-validated tumour-adjacent tissues regions as well as in tissues from age-matched chronic pancreatitis patients and from age-matched non-tumour individuals. A significantly increased number of Rep+CD68+ cells (mean around 7.4% of all cells) was observed in the tumour of pancreatic cancer patients when compared to non-cancer healthy controls (mean <1%) and when compared to tumour-adjacent tissue (mean <1%). When specifying Rep-positive cells to the fraction of M2-like, CD163-positive macrophages within the population of all macrophages, an increased number of Rep+CD68+CD163+ cells (mean around 19.5% within all macrophages) was observed in the tumour of pancreatic cancer patients when compared to healthy controls (mean 5.5% of all macrophages), indicating that a large number of Rep+ Mfs is represented by M2-like macrophages stained triple-positive with Rep, CD68 and CD163 antibodies (FIG. 9). Cell-based quantification of the same parameters for chronic pancreatitis patients indicated that the amount of Rep+CD68+ cells (mean of around 4.0%) and of Rep+CD68+CD163+ cells within all macrophages (mean of around 9.5%) localized in between the values detected for pancreatic cancer tumour tissues (with highest values, 7.4% and 19.5%, respectively) and healthy controls (showing lowest values, <1% and 5.5%, respectively). Quantification of Rep+CD68+ cells and Rep+CD68+CD163+ cells within all macrophages in tumour-adjacent tissues (mean <1% and around 5.5%) was comparable to cell counts in healthy individuals. The numbers of Rep+, CD68+ and Rep+CD68+ cells were significantly increased in the peritumour when compared to respective quantifications of stained cells in the paired tumor (medians of 6.3% vs. 0.5% Rep+, 11.0% vs. 2.5% CD68+, 2.2% vs. <0.1% Rep+CD68+ cells, respectively) (FIG. 10).

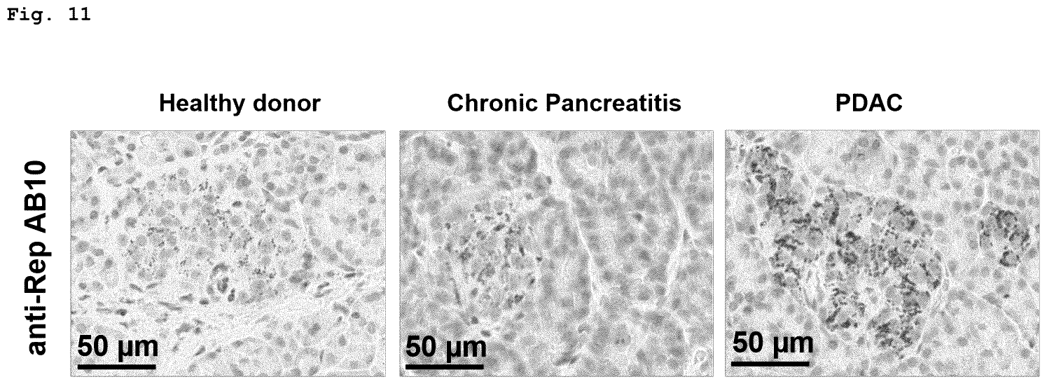

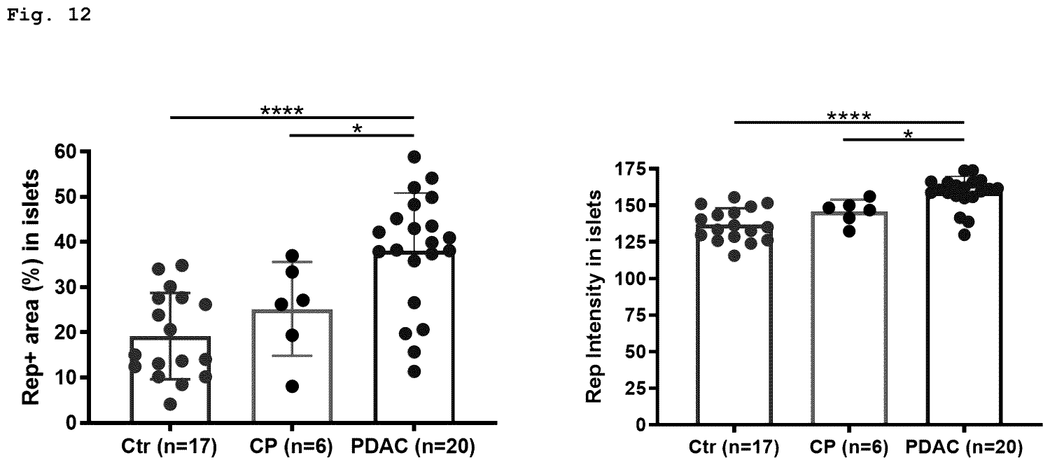

Comparison Rep-stained area in Langerhans islets and staining intensity based on semi-automated quantification for tissues of healthy donors, PDAC patients and chronic pancreatitis patients (representative DAB IHC staining examples given in FIG. 11) revealed significantly increased Rep-stained areas in Langerhans Islets of PDAC patients (approximately 38% mean) when compared to healthy controls (around 18% mean) (FIG. 12). Stained area in chronic pancreatitis (around 25% mean) was significantly smaller when compared to PDAC but still increased when compared to the group of healthy donors. The same trends were observed for Rep staining intensity, but on a generally higher level. Rep staining intensity was significantly increased in Langerhans Islets of PDAC patients (around 160 a.u., on average) when compared to healthy controls (around 135 a.u. on average). Staining intensity in chronic pancreatitis (around 140 a.u. on average) was significantly smaller when compared to PDAC, but still increased when compared to the healthy group.

The detection of Rep positivity in triple positive macrophages (Rep+CD68+CD163) was used to characterize the differentiation of Rep-positive macrophages and discriminate between a generally more M1-like (CD68+CD163−) or M2-like (CD68+CD163+) macrophage polarization. Quantification of triple positive macrophages might sharpen future diagnostic differentiation and help to identify (pathogenic) involvement of BMMF-positive macrophage populations to cancer. M1-like macrophages are expected to act pro-inflammatory (recruiting e.g. tumor-cell reactive lymphocytes) and act anti-tumorigenic while M2-like macrophages are considered pro-tumorigenic releasing cytokines and chemokines involved in recruitment of pro-tumorigenic regulatory T-cells.

The above-mentioned analyses may be seen as proof that the Rep Protein is a structural protein that can be targeted by antibodies, possibly both for preventive or therapeutic purposes.

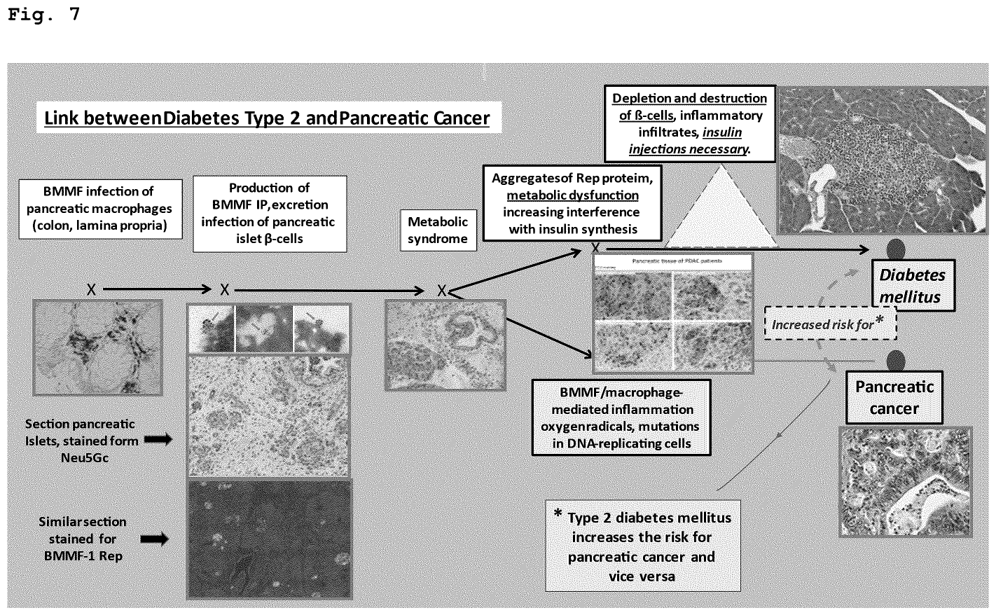

In addition, the inventors have found a link between BMMF infection and type 2 diabetes since it has been shown by the inventors that BMMF infection is prevalent in insulin-producing pancreatic β-cells in the endocrine Langerhans Islets. It results in large aggregates of BMMF Rep protein that potentially interfere with insulin production. In later stages β-cell depletion occurs leading to the dependence on insulin injections. The link between diabetes type 2 and pancreatic cancer has been exemplified in FIG. 7.

The invention is further illustrated by, but not limited to, the following examples:

### EXAMPLES

### Example 1: Detection of BMMF Protein Targets in Peritumor Pancreatic Cancer Tissue

### Tissue Staining

Peritumor pancreatic cancer tissues were obtained as FFPE section by the EPZ-Pancobank/BioMaterial Bank Heidelberg including patient consent and in accordance with the regulations of the tissue bank as approved by the Ethics Committee of Heidelberg University (Ethic votes S-708/2019 and S-083/2021).

TABLE 2

| | | | | | |
|---|---|---|---|---|---|
| | | | | Final | |
| | | | | concentration | Incubation |
| Antibody | Source | Host | Dilution | in μg/ml | time |

Primary

| | | | | | |
|---|---|---|---|---|---|
| Rep mAb | T. Bund, | mouse | 1:500 | 3.9 | 60 min at |
| #3-6 | DKFZ | | | | room |
| Rep mAb | T. Bund, | mouse | 1:500 | 3.9 | temperature |
| #10-3 | DKFZ | | | | |
| CD68 | Cell | Rabbit | 1:1000 | 0.1 | |
| | signaling | | | | |
| insulin | #76437 | mouse | 1:250 | 4 | 120 min |
| | Santa- | | | | |
| | Cruz, sc- | | | | |
| | 8033 | | | | |

For EM:

| | | | | |
|---|---|---|---|---|
| anti-mouse | DAKO, | rabbit | 1:150 | 60 min at |
| | Z0259 | | | room |
| | | | | temperature |

FFPE IHC DAB staining was performed based on the Zytomed ChemPlus (HRP) staining kit (Zytomed Systems) including epitope retrieval (30 min, 95° C.) with EDTA (pH 8.5, Sigma) as described previously (Bund et al., 2021). Rep staining with AB3 (1 h, RT) was followed by colourization with the DAB high contrast kit (Zytomed Systems) and hematoxylin counterstain. Slides were scanned using a Hamamatsu Nanozoomer slide scanner (Hamamatsu) and analyzed with NDP.view2 Plus software (Hamamatsu).

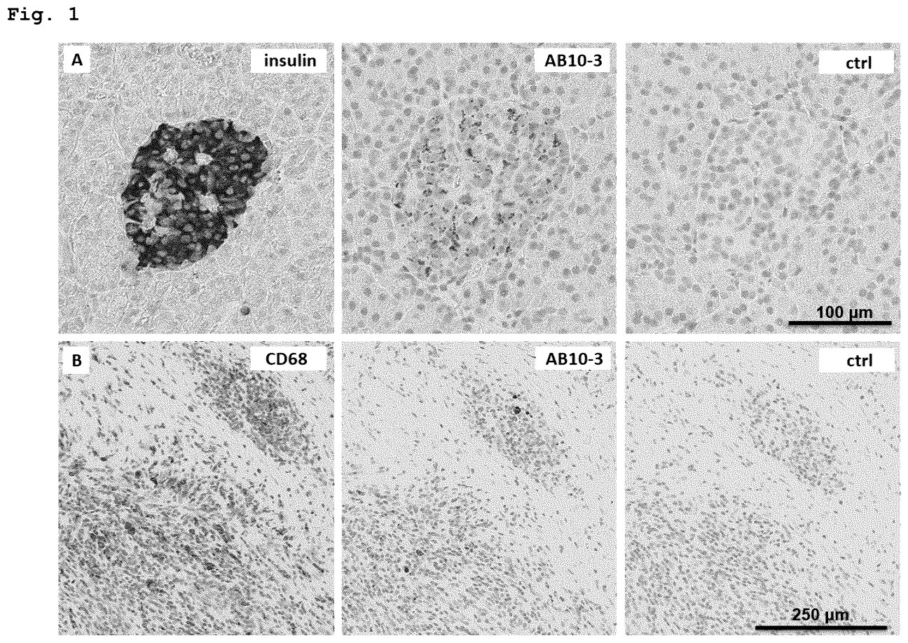

Staining of tissue PDAC tissue samples with anti-Rep antibodies (e.g. mAb 10; Table 1) shows detection of protein targets in peritumor tissue regions within pancreatic cancer patient samples. In general, the anti-Rep detection resulted in intense staining of smaller sized aggregated structures mainly within the cytoplasmic regions of insulin-positive beta-cells of the endocrine Langerhans Islands (FIG. 1, A). Additionally, a second staining pattern was observed in exocrine tissue regions. Here, a co-localization of the anti-Rep stained signals with CD68-positive macrophages, which are distributed throughout the exocrine tissue regions and in the interstitium, was observed (FIG. 1, B). The regions with highest Rep-specific antibody detection correlate with regions with highest detection levels for CD68 positive cells pointing towards a localization of the Rep-specific antigens in inflammatory tissue areas, i.e. regions with especially high levels of inflammatory monocytes, circulating macrophages, or resident tissue macrophages. No signal detection was observed in control stainings with an antibody isotype control.

### Example 2: Pre-Embedding Immuno-Electronmicroscopy (IEM)

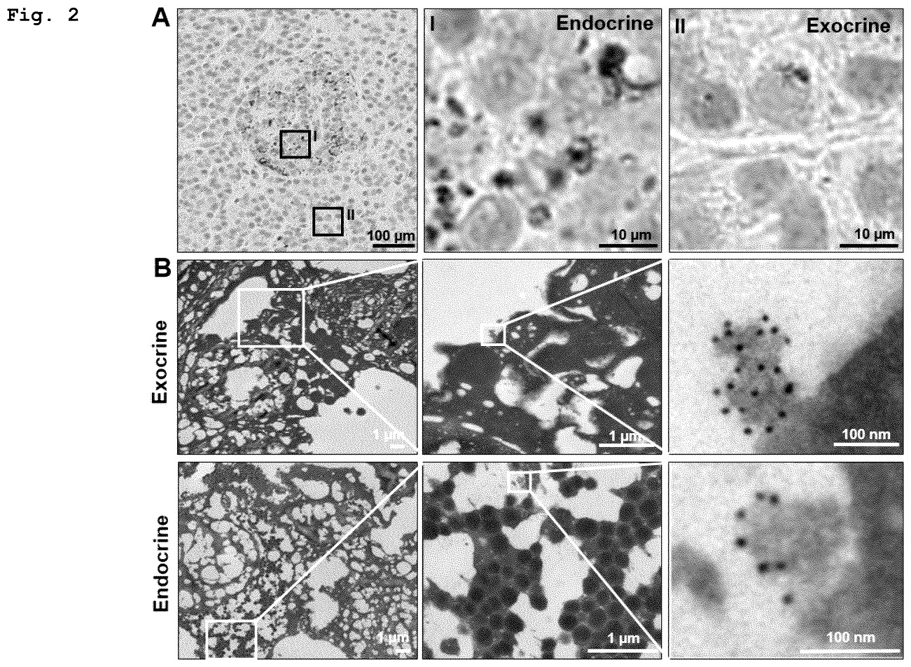

Tissue from a pancreatic cancer patient which stained positive for BMMF Rep in IHC was used to resolve the localization and physical expression of Rep targets at ultrastructural resolution. The inventors initially performed IHC DAB staining using monoclonal anti-BMMF1 Rep AB10 on one of the consecutive FFPE cuts to identify positive tissue regions (FIG. 2A). Based on Example 1, consecutive sections containing both exocrine and endocrine peritumor tissue regions were subsequently gold-labelled by pre-embedding IEM (pre-IEM) (FIG. 2B).

Therefore, standard procedures for immunolabelling of FFPE material followed by resin-embedding for subsequent ultrathin sectioning were as follows: FFPE-sections (3 μm thickness) mounted on glass slides were de-waxed with xylene and rehydrated in a graded series of ethanol. The immunoreaction was performed by incubation in 40 mM glycin (aldehyde block), 1% BSA (protein block), primary antibodies AB3 and polyclonal rabbit-anti-mouse (1:150, Z0259, Dako) as linker for the reporter Protein A-gold (CMC, nominal gold grain size 5 nm or 10 nm). Runs without primary antibody were included as reporter-only control. Sections on the glass slides were post-fixed in 2.5% glutaraldehyde, followed by 1% OsO4, dehydration through graded steps of ethanol and flat-embedding in epoxide (Serva). Ultrathin sections taken at nominal thickness 60 nm, were contrasted with uranyl and lead for direct visualization by electron microscopy.

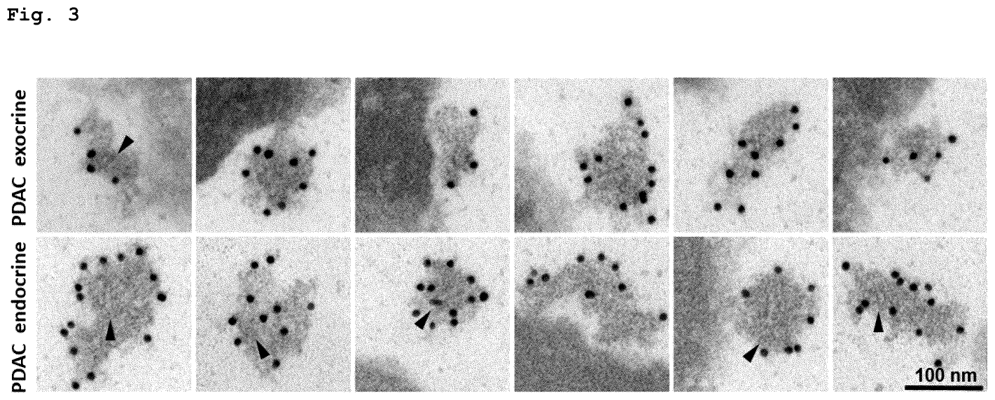

Pre-embedding IEM staining revealed exclusive gold label at high density on distinct compact particulate structures, pleomorphic in size ranging between approximately 50-200 nm both in exocrine and endocrine tissue regions. The identified pleomorphic structures vary in size and shape and appear to contain filamentous substructures which are highlighted with arrowheads (FIG. 3). Apart from these structures, gold grains did not accumulate in other regions of the tissues. The cellular context of localization was not identified due to compromised structural preservation inherent to the FFPE protocol.

### Example 3: Cryo IEM (Tokuyasu method) After Overexpression of MSBI1.176 Rep in HEK293TT for Validation of Pre-IEM

### Cell Culture and Transfection

HEK293TT cells were cultured as described previously (Buck et al. 2005, de Villiers et al. 2011) and transfected with polyethylenimine (PEI, Sigma) at 70-80% confluency in 6-well plates with 1 μg of plasmid DNA per well for overexpression of either untagged H1MSB.1 Rep or 3xFlag-H1MSB.1 Rep protein (control: mock transfection with pcDNA(-)). Overexpression plasmids were designed based on PCR-cloning of the H1MSB.1 Rep gene (cloning via BamHI/Kpnl, PCR primers: pcDNA3.1(-) H1MSB.1 Rep fwd: GC GGA TCC GCC ATG AGC GAC CTG ATC GTG AAA G (SEQ ID NO:21), rev: GC GGT ACC TCA AAA CAC GAC TCC AAA CTC TTC C (SEQ ID NO:22); pcDNA3.1(-) 3xFlag H1MSB. 1-Rep fwd: GT GGA TCC GCC ATG GAC TAC AAA GAC CAT GAC GGT GAT TAT AAA GAT CAT GAC ATC GAT TAC AAG GAT GAC GAT GAC AAG GGA GCA AGC GAC CTG ATC GTG AAA GAC AAT GC (SEQ ID NO:23), Rep rev: GA GGT ACC TCA AAA CAC GAC TCA AAC TCT TCC AGT TTA G (SEQ ID NO:24). Cells were harvested 48 hours after transfection. One part was analysed by SDS-PAGE and immunoblotting as described before (Bund et al. 2021) and the other part was fixed for cryo IEM (see below).

### Western Blot Analysis

Analysis by western blot was performed as described before (Bund et al. 2021), except that the cells were harvested after 48 h, followed by lysis in Lammli buffer. Lysates were separated by SDS-PAGE on precast gels (Sigma True-PAGE 4-20%) prior to electro-transfer to nitrocellulose. Immunodetection by western blot was performed using two primary mouse monoclonal antibodies against Rep AB3 and AB7 (DKFZ Heidelberg (Bund et al. 2021)), respectively, or anti-Flag M2 (F3165, Sigma Aldrich) and by HRP-coupled goat anti-mouse IgG secondary antibody (115-035-205, Dianova).

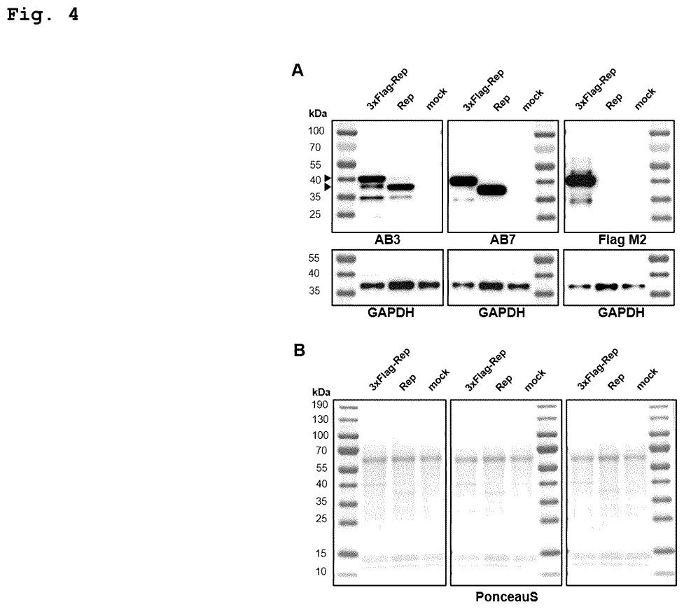

Rep protein expression was verified by WB analysis using anti-Flag, as well as anti-Rep AB3 or AB7 antibodies. Full-length Rep protein bands of a molecular weight 40 kDa and 37 kDa for 3xFlag Rep and untagged Rep, respectively, were visible. In addition, smaller molecular weight bands, which had been described in a previous study (Bund et al. 2021), stained positive (FIG. 4)

### Cryo IEM (Tokuyasu Method)

We followed the protocol of Slot & Geuze (Slot and Geuze 2007). HEK293TT cells transfected with the respective plasmids were fixed in buffered aldehyde (2% formaldehyde, 0.2% glutaraldehyde, 100 mM phosphate buffer, pH 7.2) and embedded in 10% gelatin. Vibratome-sections (200 μm thickness) of patient biopsies or blocks of gelatin-embedded cells were perfused with 2.3 M sucrose in 50 mM phosphate buffer and frozen in liquid nitrogen. Cryo sections with nominal thickness of 60 nm were cut at 160 K with an UCT7/FC7 cryo-ultramicrotome (Leica). Immunoreactions were performed on ice after buffer wash and incubation in 40 mM glycine and 1% BSA. To enable binding of protein A-gold reporter (1:50, CMC, nominal size of gold grains: 5 nm or 10 nm), a polyclonal rabbit anti-mouse antibody (1:150, Z0259, DAKO) was used to bridge the primary antibodies of murine origin. Control samples included a reporter-only (without primary antibody), or staining either with primary antibody isotype IgG1 (MG1-45, Biolegend) or anti-Flag.

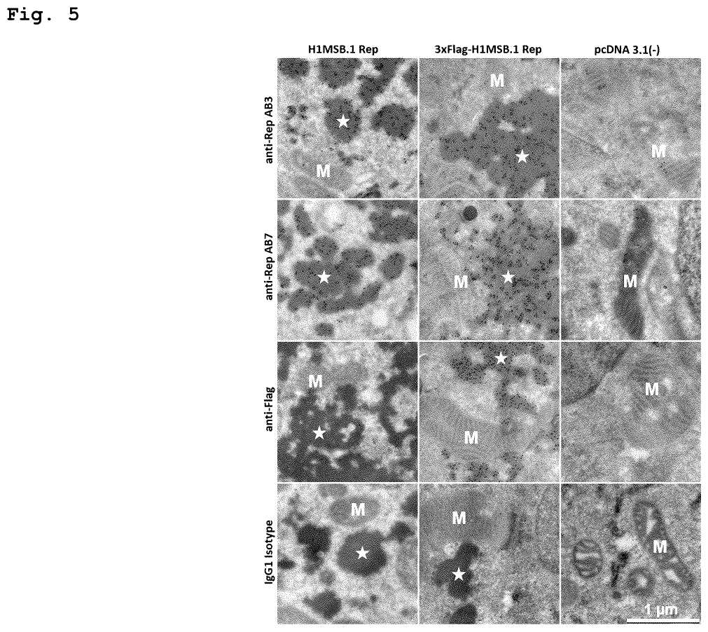

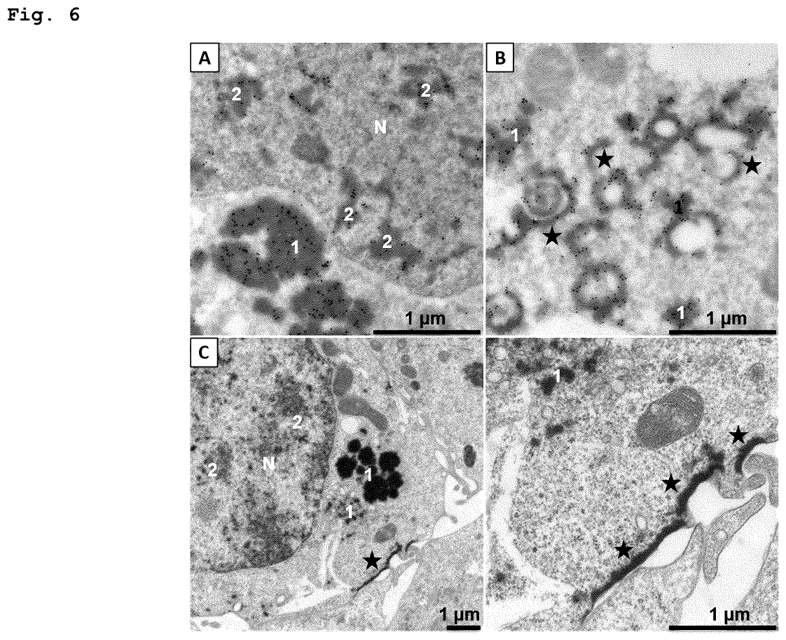

Overexpression of both constructs led to the formation of large segregation complexes in the cytoplasm of transfected cells (FIG. 5). These complexes stained positive (with anti-Rep AB3, AB7 and anti-Flag, respectively), whereas the residual cytoplasm remained gold label-free. Untagged H1MSB.1 Rep segregated in addition in a nucleoplasmic location (FIG. 6A) and showed an association with membranes (FIG. 6B). An isotype antibody did not react with the target structures (FIG. 5). Transfection with a pcDNA3.1(-) vector (no Rep insert) did not result in segregation structures.

### Resin TEM (Without Immunolabelling)

Transfected HEK293TT cells expressing H1MSB.1 Rep and cultivated on punched Aklar-Fluoropolymer films (EMS) were resin-embedded for ultrathin sectioning according to standard procedures. This included primary fixation in buffered aldehyde (4% formaldehyde, 2% glutaraldehyde, 1 mM CaCl2, 1 mM MgCl2 in 100 mM Na-cacodylate, pH 7.2), post-fixation in buffered 1% osmium tetroxide and en-block staining in 1% uranylacetate, dehydration in graded steps of ethanol and embedding in epoxide (glycidether, NMA, DDSA; Serva). Ultrathin sections, with nominal thickness 60 nm and contrast-stained with lead-citrate and uranylacetate, were analysed with EM 910 at 80 kV (Carl Zeiss) and micrographs taken using a slow scan CCD camera (TRS).

Overexpression of non-tagged MSBI1.176 Rep led to the formation of large segregation complexes in the cytoplasm of transfected cells not observed after mock transfection with pcDNA3.1(-). The segregation structures showed an association with membranes (see asterisks, FIG. 6C).

### Example 4: Cell-Based Quantification of Rep-, CD68- and CD163-Positive Cells in Tumour and Peritumour Tissues of Pancreatic Ductal Adenocarcinoma Patients, Chronic Pancreatitis Patients and Non-Cancer Controls by Immunofluorescence Microscopy

### Tissue Staining and Tissue Analysis

Tissues from pancreatic cancer patients, patients with chronic pancreatitis and tissues from healthy transplant donors were obtained as FFPE sections the PancoBank of European Pancreas Center (EPZ) Heidelberg, Germany in accordance with the regulations of the Tissue Bank which include patient consent and approval by the Ethics committee of the Heidelberg University (ethics vote S-708/2019) and in full concordance to the standards set by the Declaration of Helsinki. FFPE sections were stained with primary anti-BMMF1 Rep (monoclonal AB3 and AB10, DKFZ Heidelberg, 1:1 mix), anti-CD68 (Cell signaling #76437) and anti-CD163 (Novusbio, NB110-40686) antibodies overnight at room temperature in a dilution of 1:250 (Rep), 1:500 (CD68) and 1:200 (CD163) as described in Bund et al., 2021. Secondary goat anti-mouse Alexa Fluor 594 (Invitrogen #A11032), goat anti-rabbit Alexa Fluor 488 (Invitrogen, #A11034) and donkey anti-mouse Alexa Fluor 647 (Invitrogen #A31571) were incubated for 60 min at room temperature. For negative control staining, PBT diluent containing PBS, BSA, Tween20 and sodiumazide was used. Slides were scanned with a Hamamatsu Nanozoomer slide scanner (Hamamatsu) and analyzed with NDP.view2 Plus software (Hamamatsu).

Semi-automated quantification of Rep, CD68, CD163 staining of individual FFPE tissues was performed with an in-house developed script for ImageJ/Fiji (v1.52) based on signal intensity of DAPI nuclear and antibody staining of cells in digitalized scans after Rep/CD68/CD163 co-immunodetection. Cell density was measured in 8-12 randomly selected microscopic areas (around 15,000 cells per sample, in total).

The data was represented as % average number (mean) of Rep, CD68, CD163 positive cells and/or its combinations normalized either to total number of nuclei (or total number of CD68+ macrophages) as described in Bund et al., 2021 and Nikitina et al, 2023 for similar quantification in colorectal cancer context.

The results are shown in FIGS. 9 and 10.

TABLE 3

| | | | | Final | |
|---|---|---|---|---|---|
| | | | | concentration | Incubation |
| Antibody | Source | Host | Dilution | in μg/ml | time |
| Rep mAb | T. Bund, | mouse | 1:1000 | 2 | 60 min at |
| #3-6 | DKFZ | | | | room |
| Rep mAb | T. Bund, | mouse | 1:1000 | 2 | temperature |
| #10-3 | DKFZ | | | | |
| CD68 | Cell | Rabbit | 1:1000 | 0.1 | |
| | signaling | | | | |
| | #76437 | | | | |
| CD163 | Novusbio, | Mouse | 1:200 | 5 | |
| | Centennial, | | | | |
| | CO, USA, | | | | |
| | NB110-40686 | | | | |
| Insulin | Santa Cruz | Mouse | 1:200 | 1 | 180 min at |
| | Bio - | | | | room |
| | technology, | | | | temperature |
| | Dallas, | | | | |
| | Texas, sc- | | | | |
| | 8033 | | | | |

### Example 5: Semi-Automated Quantification of Rep+Insulin+ Cells in Tumour and Peritumour Tissues of Pancreatic Ductal Adenocarcinoma Patients, Chronic Pancreatitis Patients and Non-Cancer Controls by Immunofluorescence Microscopy

Tissue Staining and Tissue Analysis

Tissues (same as described in Example 4) were stained with primary anti-BMMF1 Rep (monoclonal AB10, DKFZ Heidelberg anti-BMMF1 Rep (monoclonal AB10, DKFZ Heidelberg, 1:500 dilution, 30 min incubation at room temperature) and anti-Insulin antibody (in consecutive tissue section to allocate β-cell island, Santa Cruz Biotechnology, Dallas, Texas, sc-8033, mouse, 1:200, 180 min at RT). Secondary rabbit anti-mouse (Abcam, Cambridge, UK, #125904) was incubated for 20 min at room temperature. Detection was performed by using Bond Polymer Refine Detection Kit (Leica Biosystems, #DS9800) including DAB chromogen and hematoxylin counterstain. Both DAB brightfield and immunofluorescent slides were scanned with a Hamamatsu Nanozoomer slide scanner (Hamamatsu, Shizuoka, Japan) and analyzed with ndp.view2 plus software (Hamamatsu).

For the analyses of Rep antibody-stained positive area and staining intensity of pancreatic islets, semi-automated quantification Rep staining was performed with an in-house developed script for ImageJ/Fiji (v1.52) based on DAB signal area and mean intensity in each selected islets of tissue. 20-40 islets per tissue (maximum number available) were included into analyses. The data was represented as % Rep-stained area in Langerhans islets (mean) and Rep staining intensity in Langerhans islets as described in Nikitina et al., 2022.

The results are shown in FIG. 11 (representative DAB IHC tissue staining) and FIG. 12.

SEQUENCE SUMMARY

| SEQ | |
|---|---|
| ID | |
| NO | SEQUENCE |
| 1 | Amino acid sequence of Rep protein encoded by MSBI1.176 |
| | MSDLIVKDNALMNASYNLALVEQRLILLAIIEARETGKGINANDPLTVHASSYINQFN |
| | VERHTAYQALKDACKDLFARQFSYQEKRERGRINITSRWVSQIGYMDDTATVEIIFAP |
| | AVVPLITRLEEQFTQYDIEQISGLSSAYAVRMYELLICWRSTGKTPIIELDEFRKRIG |
| | VLDTEYTRTDNLKMRVIELALKQINEHTDITASYEQHKKGRVITGFSFKFKHKKQNSD |
| | KTPKNSDSSPRIVKHSQIPTNIVKQPENAKMSDLEHRASRVTGEIMRNRLSDRFKQGD |
| | ESAIDMMKRIQSEIITDAIADQWESKLEEFGVVF |
| 2 | Amino acid sequence of Rep peptide fragment |
| | EARETGKGINANDPLTVH |
| 3 | Amino acid sequence of Rep peptide fragment |
| | KQINEHTDITASYEQHKKGRT |
| 4 | His-Tag (with two neutral stuffer amino acids) |
| | GAHHHHHH |
| 5 | T7-Tag |
| | MASMTGGQQMG |
| 6 | FLAG-Tag |
| | DYKDDDDK |
| 7 | Strep-II-Tag |
| | WSHPQFEK |
| 8 | Amino acid sequence of Rep protein encoded by MSBI2.176 |
| | MSKLVVKDNALMNASYNLDLVEQRLILLAIIEARESGKGINANDPLTVHA |
| | ESYINQFGVHRVTAYQALKDACDNLFARQFSYQSKSEKGNIQNHRSRWVS |
| | EIIYIDTEATVKIIFAPAIVPLITRLEEQFTKYDIEQISDLSSAYAIRLY |
| | ELLICWRSTGKTPIIGLGEFRNRVGVLDSEYHRIAHLKERVIEHSIKQIN |
| | EHTDITATYEQHKKGRTITGFSFKFKQKKPKQAEIATETPKTATNDPDTT |
| | KPLTEPQIAKYSMILCKLGSISDLSNFPDYPAFANWIGNILRNPEKADEQ |
| | IAKRIFTALKTETDYSKKN |
| 9 | MSBI.1 specific epitope |
| | NRLSDRF |
| 10 | Amino acid sequence of Rep protein encoded by CMI1.252 |
| | MSDLIVKDNALMNASYNLALVEQRLILLAILEARETGKGINANDPLTVHASSYINQFN |
| | VERHTAYQALKDACKDLFARQFSYQEKRERGRINITSRWVSQIGYMDDTATVEIIFAP |
| | AVVPLITRLEEQFTQYDIEQISELSSAYAVRLYELLICWRSTGKTPIIDLTEFRKRLG |
| | VLDTEYTRTDNLKMRVIELGLKQINEHTDITASYEQHKKGRTITGFSFKFKQKKKTGA |
| | EMPKNSDSSPHIEKPSQIPANIAKQPENAKKDDLGHRASKITGLIMSNGLADRFKRGD |
| | ESVIDMMKRIKEEITTDTTADQWENKLEEFGVIFQS |
| 11 | Amino acid sequence of Rep protein encoded by CMI2.214 |
| | MSDLIVKDNALMNASYNLDLVEQRLILLAILEARETGKGINANDPLTVHAESYINQFG |
| | VARQTAYQALKDACKDLFARQFSYQEKRERGRANITSRWVSQIAYIDETATVEVIFAP |
| | AVVPLITRLEEQFTQYDIEQISGLSSAYAVRLYELLICWRSTGKTPVIELAEFRKRLG |
| | VLNDEYTRSDNFKKWIIENPIKQINEHTDITASYEQHKKGRTITGFSFKFKQKKKTEP |
| | ETPKNSDSSQRIEKPSQIPANIVKQPENANLSDLQHRASKITGLIMSNRLSDRFKQGD |
| | ESIMQMMARIQSEITTDSIADQWQSKLEEFGVVF |
| 12 | Amino acid sequence of Rep protein encoded by CMI3.168 |
| | MSDLIVKDNALMNASYNLALVEQRLILLAILEARETGKGINANDPLTVHASSYINQFN |
| | VERHTAYQALKDACKDLFARQFSYQEKRERGRANITSRWVSQIAYIDETATVEVIFAP |
| | AVVPLITRLEEQFTQYDIEQISGLSSAYAVRLYELLICWRTTGKTPVLDLTEFRKRLG |
| | VLDTEYTRTDNLKMRVIEQSLKQINKHTDITASYEQHKKGRTITGFSFKFKQKKKTEP |
| | ETPKNNDSGVSKPKTVEIPAEVVKQPKNTNLSDLEKRVRMITGAIAKNNLASRFQHGN |
| | ESPLDMMKRIQSEITSDETADLWQNKLESMGVVF |
| 13 | DNA sequence MSBI1 Rep codon-optimized |
| | ATGAGCGACCTGATCGTGAAAGACAATGCCCTGATGAACGCCTCCTACAACCTGGCAC |
| | TGGTCGAACAGAGACTGATTCTGCTGGCTATCATCGAGGCAAGGGAGACCGGCAAGGG |
| | CATCAACGCCAATGACCCCCTGACAGTGCACGCCAGCTCCTACATCAACCAGTTTAAT |
| | GTGGAGCGCCACACCGCCTATCAGGCCCTGAAGGACGCCTGCAAGGATCTGTTTGCCC |
| | GGCAGTTCAGCTACCAGGAGAAGCGGGAGAGAGGCAGGATCAACATCACAAGCAGATG |
| | GGTGTCCCAGATCGGCTATATGGACGATACCGCCACAGTGGAGATCATCTTTGCACCA |
| | GCAGTGGTGCCTCTGATCACCAGGCTGGAGGAGCAGTTCACACAGTACGACATCGAGC |
| | AGATCTCCGGACTGTCTAGCGCCTACGCCGTGCGCATGTATGAGCTGCTGATCTGTTG |
| | GCGGTCTACCGGCAAGACACCTATCATCGAGCTGGATGAGTTCCGCAAGCGGATCGGC |
| | GTGCTGGACACCGAGTACACCAGAACAGATAACCTGAAGATGAGAGTGATCGAGCTGG |
| | CCCTGAAGCAGATCAATGAGCACACCGATATCACAGCCTCTTATGAGCAGCACAAGAA |
| | GGGCCGCGTGATCACCGGCTTCAGCTTTAAGTTCAAGCACAAGAAGCAGAACTCTGAC |
| | AAGACACCAAAGAATAGCGATTCCTCTCCCCGGATCGTGAAGCACAGCCAGATCCCTA |
| | CCAACATCGTGAAGCAGCCAGAGAATGCCAAGATGTCCGACCTGGAGCACAGGGCATC |
| | TAGGGTGACAGGCGAGATCATGAGAAATAGGCTGAGCGATCGGTTCAAGCAGGGCGAC |
| | GAGTCCGCCATCGATATGATGAAGAGAATCCAGTCCGAGATCATCACCGACGCCATCG |
| | CCGATCAGTGGGAATCTAAACTGGAAGAGTTTGGAGTCGTGTTTGGAGCACATCACCA |
| | TCATCATCACTGA |
| 14 | Protein sequence MSBI1 Rep codon-optimized |
| | MSDLIVKDNALMNASYNLALVEQRLILLAIIEARETGKGINANDPLTVHASSYINQFN |
| | VERHTAYQALKDACKDLFARQFSYQEKRERGRINITSRWVSQIGYMDDTATVEIIFAP |
| | AVVPLITRLEEQFTQYDIEQISGLSSAYAVRMYELLICWRSTGKTPIIELDEFRKRIG |
| | VLDTEYTRTDNLKMRVIELALKQINEHTDITASYEQHKKGRVITGFSFKFKHKKONSD |
| | KTPKNSDSSPRIVKHSQIPTNIVKOPENAKMSDLEHRASRVTGEIMRNRLSDRFKOGD |
| | ESAIDMMKRIQSEIITDAIADQWESKLEEFGVVEGA |
| 15 | DNA sequence MSBI1 Rep wild-type |
| | ATGAGCGATTTAATAGTAAAAGATAACGCCCTAATGAATGCTAGTTATAACTTAGCTT |
| | TGGTTGAACAGAGGTTAATTCTATTAGCAATCATAGAAGCGAGAGAAACAGGCAAAGG |
| | GATTAATGCCAATGATCCTTTAACAGTTCATGCAAGTAGCTATATCAATCAATTTAAC |
| | GTAGAAAGGCATACGGCATATCAAGCCCTCAAAGATGCTTGTAAAGACTTGTTTGCCC |
| | GTCAATTCAGTTACCAAGAAAAGCGAGAACGAGGACGAATTAATATTACAAGTCGATG |
| | GGTTTCGCAAATTGGCTATATGGACGATACAGCAACCGTTGAGATTATTTTTGCCCCT |
| | GCGGTTGTTCCTCTGATTACACGGCTAGAGGAACAGTTCACCCAGTACGATATTGAGC |
| | AAATTAGCGGTTTATCGAGTGCATATGCTGTTCGTATGTACGAACTGCTGATTTGTTG |
| | GCGTAGCACAGGCAAAACACCAATTATTGAGCTAGACGAGTTTAGAAAGCGAATAGGT |
| | GTTTTAGATACTGAATACACTAGAACAGATAATTTAAAGATGCGAGTTATTGAATTAG |
| | CCCTAAAACAAATCAACGAACATACAGACATCACAGCAAGCTATGAACAACACAAAAA |
| | AGGGCGAGTGATTACAGGATTCTCATTCAAGTTTAAGCACAAGAAACAAAACAGCGAT |
| | AAAACGCCAAAAAATAGCGATTCTAGCCCACGTATCGTAAAACATAGTCAAATCCCTC |
| | CAACATTGTAAAACAGCCTGAAAACGCCAAAATGAGCGATTTAGAACATAGAGCGAGC |
| | CGTGTTACAGGGGAAATAATGCGAAATCGTCTGTCAGATCGGTTTAAACAAGGCGATG |
| | AATCAGCAATCGACATGATGAAACGTATTCAAAGTGAAATAATAACCGATGCAATAGC |
| | AGACCAGTGGGAAAGCAAACTGGAGGAGTTTGGCGTGGTTTTTTAG |
| 16 | Amino acid sequence of Rep protein encoded by CMI4.158 |
| | MSDLIVKDNALMNASYNLALVEQRLILLAILEARETGKGINAND |
| | PLTVHANSYINQFNVERHTAYQALKDACKDLFARQFSYQEKRERGRINITSRWVSQIG |
| | YMDDTATVEVIFAPAVVPLITRLEEQFTQYDIEQISGLSSAYAVRLYELLICWRSTGK |
| | TPVIELAEFRKRIGVLDTEYTRTDNLKMRVIELALKQINEHTDITASYEQHKKGRTIT |
| | GFSFKFKQKKKTELETPKNSDSSPHIEKPSQIPTNIVKQPENAKKDDLGHRASKITGL |
| | IMSNGLADRFKRGDESVMNMMKRIKEEITTDATADLWQNKLESMVWFFSHD |
| 17 | Amino acid sequence of Rep protein encoded by HCBI3 |
| | MYRWFMSDLIVKDNALMNASYNLDLVEQRLVLLAILEARESGKGINANDP |
| | LTVHAESYINQFGVARQTAYQALKGACKDLFARQFSYQEKRERGRANITS |
| | RWVSQIAYIDETATVEVIFAPAVVPLITRLEEQFSKYDIEQISSLSSAYA |
| | VRLYELLICWRTTGKTPVIDLTEFRKRIGVLDTEYQRMDVFKRGVLELAL |
| | KQVNEHTDITATYEQHKKGRVITGFSFKFKQKKPKQAEIATETPKTATTD |
| | LATTKPLTEPQIAKYSMILCKLGSISDLGNFPDYPTFANWIANILRTPEK |
| | ADEQIAKRIFTALKTETDYSKKN |
| 18 | Amino acid sequence of Rep protein encoded by HCBI4 |
| | MKNGLVVKDNALMNASYNLEVTEQRLILLAIISARETGQGITSD |
| | SKLEIHASDYAIQFSVAKETAYEALKSAVNNLFERQFSFREETKKGTGIVRSRWVSRI |
| | KYIDDAAILEITFAPDVVPLITRLEEHFTSYQIKQIAQLTSKYAIRLYELLIAWRTTG |
| | KVPELELSEFRNRLGIASNEYTAMNNFKSRVLEPSIKQINEHTDITVTYEQHKKGRTI |
| | TGFSFRFKQKQQAKKIETNRDPNTPDFFIKMTDAQRHLFANKMSEMPEMGKYSQGTES |
| | YQQFAIRIADMLLQPEKFRELYPILEKAGFKE |
| 19 | Amino acid sequence of Rep protein encoded by C1MI.3M.1 |
| | MSELIVKDNALMNASYNLDLVEQRLILLAILEARESGKGINANDPLTVHA |
| | ESYINQFGVHRTTAYQALKDACKDLFARQFSYQEKRERGRANITSRWVSQ |
| | IAYIDETGTVEVIFAPAVVPLITRLEEQFTKYDIEQISGLSSAYAVRLYE |
| | LLICWRTTGKTPIIDLTEFRKRIGVLDTEYQRMDVFKRGVLELALKQVNE |
| | HTDITASYEQHKKGRVITGFSFKFKQKKPKQAEIATETPKTATNDPDTTK |
| | PLTEPQIAKYSMILCKLGSISDLSNFPDYPAFANWIGNILRNPEKADEQI |
| | AKRIFTALKTETDYSKKN |
| 20 | Amino acid sequence of Rep protein encoded by C1MI.9M.1: |
| | MRLMSELVVKDNALMNASYNLDLVEQRLILLAIVEARESGKGIN |
| | ANDPLTVHAESYINQFGVARQTAYQALKDACDDLFARQFSYQEINERGNTENVRSRWV |
| | SEVRYVDAEATVKLIFAPIVIPLITRLEERFTQYEMKQISELSTGYAIRLYELLICWR |
| | ATGKTPVIELADFRQRMGVLDSEYQRMYDLKKYVLEPSLKQINEHTDITATYEQHKKG |
| | RVITGFSFKFKQKKPKQAEIATETPKTATNDQNTIKPLTEPQIAKYSMILCKLGSISD |
| | LSNFPDYPSFANWIANILRTPEKADEQAAKRIFTALKTETDYSKKN |

### REFERENCES

Eilebrecht, S., et al. (2018), “Expression and replication of virus-like DNA in human cells”, Scientific Reports 8:2851
Funk, M., et al. (2014). “Isolation of protein-associated circular DNA from healthy cattle serum”. Genome Announc 2(4)
Giraldo, R., et al. (2011). “RepA-WH1 prionoid: a synthetic amyloid proteinopathy in a minimalist host.” Prion 5(2):60-64
Gunst, K., et al. (2014). “Isolation of bacterial plasmid-related replication-associated cirular DNA from a serum sample of a multiple sclerosis patient.” Genome Announc 2(4).
Lamberto, I., et al. (2014). “Mycovirus-like DNA virus sequences from cattle serum and human brain and serum samples from multiple sclerosis patients.” Genome Announc 2(4).
Manuelidis L., 2011. “Nuclease resistant circular DNAs co-purify with infectivity in scrapie and CJD”. J. Neurovirol. 17:131-145.
Torreira, E., et al. (2015). “Amyloidogenesis of bacterial prionoid RepA-WH1 recaptiulates dimer to monomer transitions of RepA in DNA replication initiation.” Structure 23 (1): 183-189
Whitley, C., et al. (2014). “Novel replication-competent cirulara DNA molecules from healthy cattle serum and milk and multiple sclerosis-affected human brain tissue.” Genome Announc 2(4).
zur Hausen, H., Bund, T., de Villiers, E.-M. (2017). “Infectious agents in bovine red meat and milk and their potential role in cancer and other chronic diseases.” Curr. Top. Microbiol. Immunol., Volume 407, 83-116.
Bund, T., E. Nikitina, D. Chakraborty, C. Ernst, K. Gunst, B. Boneva, C. Tessmer, N. Volk, A. Brobeil, A. Weber, M. Heikenwalder, H. zur Hausen and E. M. de Villiers (2021). Analysis of chronic inflammatory lesions of the colon for BMMF Rep antigen expression and CD68 macrophage interactions. Proc Natl Acad Sci USA 118(12):e2025830118. doi: 10.1073/pnas.2025830118.
Slot, J. W. and H. J. Geuze (2007). Cryosectioning and immunolabeling. Nat Protoc 2(10):2480-2491.
Falida, K., S. Eilebrecht, K. Gunst, H. zur Hausen and E. M. de Villiers (2017). Isolation of Two Virus-Like Circular DNAs from Commercially Available Milk Samples. Genome Announc 5(17):e00266-17. doi: 10.1128/genomeA.00266-17.
König, M. T., R. Fux, E. Link, G. Sutter, E. Märtlbauer and A. Didier (2021a). Circular Rep-Encoding Single-Stranded DNA Sequences in Milk from Water Buffaloes (Bubalus arnee f. bubalis). Viruses 13(6):1088. doi: 10.3390/v13061088.
König, M. T., R. Fux, E. Link, G. Sutter, E. Märtlbauer and A. Didier (2021b). Identification and Characterization of Circular Single-Stranded DNA Genomes in Sheep and Goat Milk. Viruses 13(11):2176. doi: 10.3390/v13112176.
De Villiers and zur Hausen (2021), Bovine Meat and Milk Factors (BMMFs): Their proposed role in common human cancers and type 2 diabetes mellitus, Cancers (Basel), 13(21):5407
De Villiers, E.M., Gunst, K., Chakraborty D., Ernst, C., Bund, T., zur Hausen, H., (2019), A specific class of infectious agents isolated from bovine serum an diary products and peritumoral colon cancer tissue, Emerging Microbes and Infections 8(1), 1205-1218
Nikitina, E., Burk-Körner, A., Wiesenfarth, M., Alwers, E., Heide, D., Tessmer, C., Ernst, C., Krunic, D., Schrotz-King, P., Chang-Claude, J., von Winterfeld, M., Herpel, E., Brobeil, A., Brenner, H., Heikenwalder, M., Hoffmeister, M., Kopp-Schneider, A., Bund, T., Bovine meat and milk factor protein expression in tumor-free mucosa of colorectal cancer patients coincides with macrophages and might interfere with patient survival. Molecular Oncology, 2023. DOI: 10.1002/1878-0261.13390
Nikitina, E., Alikhanyan, K., Nessling, M., Richter, K., Kaden, S., Ernst, C., Seitz, S., Chuprikova, L., Haefele, L., Gunst, K., Rahbari, N., Birgin, E., Rasbach, E., Rahbari, M., Brobeil, A., Schenk, M., Büchler, M., de Villiers, E.-M., Bund, T.*, zur Hausen, H. Structural Expression of BMMF in tissues of colorectal, lung and pancreatic cancer patients. International Journal of Cancer, 2022. DOI: 10.1002/ijc.34374

1. Use of Bovine Meat and Milk Factor Group 1 (BMMF1) Rep Protein as a diagnostic marker for pancreatic cancer.

2. The use of claim 1, wherein the Rep protein is a MSBI1 genome-encoded Rep protein (MSBI1 Rep), a MSBI2 genome-encoded Rep protein (MSBI2 Rep), a CMI1 genome-encoded Rep protein (CMI1 Rep), a CMI2 genome-encoded Rep protein (CMI2 Rep), CMI3 genome-encoded Rep protein (CMI3 Rep) or CMI4 genome-encoded Rep protein (CMI4 Rep).

3. A method for providing a diagnosis or predisposition for pancreatic cancer in a subject, comprising the step of
detecting Rep protein in a sample obtained from a subject by anti-Rep antibodies that bind to an epitope comprised by SEQ ID NO:2 or SEQ ID NO:3.

4. The method of claim 3, wherein the antibody specific for Rep protein binds to an epitope that is within an amino acid sequence selected from the group consisting of amino acids from 1 to 136, from 137 to 229 and from 230 to 324 of SEQ ID NO:1.

5. The method of claim 3, wherein the sample from a subject is selected from the group consisting of a cancerous pancreatic tissue, peripheral tissue surrounding the cancerous tissue, (benign) cysts.

6. The method of claim 3, wherein additionally CD68 positive cells are detected in the sample by an anti-CD68 antibody.

7. The method of claim 3, wherein additionally CD163 positive cells are detected in the sample by an anti-CD163 antibody.

8. The method of claim 3, wherein additionally insulin positive cells are detected in the sample by an anti-insulin antibody.

9. Use of Bovine Meat and Milk Factor Group 1 (BMMF1) Rep Protein as a diagnostic marker for diabetes.
